(19) United States
(12) Patent Application Publication
(10) Pub. No.: US 2020/0093545 A1
(43) Pub. Date: Mar. 26, 2020

(54) CONTROL APPARATUS, CONTROL METHOD, AND MEDICAL OBSERVATION SYSTEM

(71) Applicant: Sony Corporation, Tokyo (JP)

(72) Inventors: Tatsumi SAKAGUCHI, Kanagawa (JP); Yoshio SOMA, Kanagawa (JP)

(73) Assignee: Sony Corporation, Tokyo (JP)

(21) Appl. No.: 16/469,151

(22) PCT Filed: Oct. 10, 2017
(86) PCT No.: PCT/JP2017/036587

§ 371 Date: Jun. 13, 2019

(30) Foreign Application Priority Data
Dec. 20, 2016 (JP) 2016-246923

(51) Int. Cl. A61B 34/20; H04N 5/262; G06T 7/70; H04N 5/232; H04N 5/225; A61B 1/247; A61B 1/045

(52) U.S. Cl. CPC A61B 34/20; H04N 5/2628; G06T 7/70; H04N 5/23299; A61B 2034/2065; H04N 5/2253; A61B 1/247; A61B 1/045; G06T 2207/30036; H04N 5/23222

(57) ABSTRACT

[Object] To provide a control apparatus, a control method, and a medical observation system.

[Solution] A control apparatus including: an acquisition unit configured to acquire dental mirror detection information from a picked up image acquired by an image pickup apparatus; and a control unit configured to, on the basis of the dental mirror detection information, perform at least one of image processing on the picked up image or arm control processing that controls an arm unit that supports the image pickup apparatus.

### TECHNICAL FIELD

The present disclosure relates to a control apparatus, a control method, and a medical observation system.

### BACKGROUND ART

In a dental treatment, it is difficult in some cases to directly look at an affected area, and while observing a mirror reflection image of a dental mirror grasped by one hand, a surgeon (dentist), for example, may perform a treatment using a surgical instrument grasped by the other hand. Further. Patent Literature 1 discloses a system in which a video camera is mounted on the handle portion of a dental mirror, and a mirror reflection image is picked up by the video camera.

On the other hand, in recent years, a video microscope is being used in a dental treatment. In order to obtain a desired field of view (display image) in a video microscope, a surgeon, for example, can determine the field of view by determining the position and the angle of the video microscope. Thereafter, the surgeon, for example, can adjust the position and the angle of the dental mirror in accordance with the obtained field of view to perform a dental treatment while observing an affected area.

### CITATION LIST

### Patent Literature

Patent Literature 1: JP 2005-066284A

### DISCLOSURE OF INVENTION

### Technical Problem

However, as described above, the both hands of the surgeon during a dental treatment are occupied by the dental mirror and the surgical instrument, so that it is difficult in some cases to change the position and the angle of the video microscope in order to observe the affected area more freely, for example.

Therefore, the present disclosure proposes a control apparatus, a control method, and a medical observation system that are novel and improved, and enable an affected area (observation target) to be observed more freely in a dental treatment through use of a video microscope and a dental mirror.

### Solution to Problem

According to the present disclosure, there is provided a control apparatus including: an acquisition unit configured to acquire dental mirror detection information from a picked up image acquired by an image pickup apparatus; and a control unit configured to, on the basis of the dental mirror detection information, perform at least one of image processing on the picked up image or arm control processing that controls an arm unit that supports the image pickup apparatus.

In addition, according to the present disclosure, there is provided a control method including: acquiring dental mirror detection information from a picked up image acquired by an image pickup apparatus; and on the basis of the dental mirror detection information, performing, with a processor, at least one of image processing on the picked up image or arm control processing that controls an arm unit that 30 supports the image pickup apparatus.

In addition, according to the present disclosure, there is provided a medical observation system including: an image pickup apparatus configured to acquire a picked up image; and a control apparatus including an acquisition unit configured to acquire dental mirror detection information from the picked up image, and a control unit configured to, on the basis of the dental mirror detection information, perform at least one of image processing on the picked up image or arm control processing that controls an arm unit that supports the image pickup apparatus.

### Advantageous Effects of Invention

According to the present disclosure as described above, it is possible to observe an affected area (observation target) more freely in a dental treatment through use of a video microscope and a dental mirror.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is a view depicting an example of a schematic configuration of a medical observation system 5300 to which an embodiment of the present disclosure may be applied.

FIG. 2 is a view illustrating a state of surgery in which the medical observation system 5300 is used.

FIG. 3 is a block diagram illustrating an example of a functional configuration of a control apparatus 1-1 according to a first embodiment of the present disclosure.

FIG. 4 includes explanatory diagrams illustrating an example of cutting out processing and image enlarging processing performed by an image processing unit 112 according to the embodiment.

FIG. 5 is a flowchart illustrating an exemplary operation of the control apparatus 1-1 according to the embodiment.

FIG. 6 is a block diagram illustrating an example of a functional configuration of a control apparatus 1-2 according to a second embodiment of the present disclosure.

FIG. 7 is a flowchart illustrating an example of an operation of the control apparatus 1-2 according to the embodiment.

FIG. 8 is a flowchart illustrating another example of an operation of the control apparatus 1-2 according to the embodiment.

FIG. 9 is a flowchart illustrating another example of an operation of the control apparatus 1-2 according to the embodiment.

FIG. 10 is a flowchart illustrating another example of an operation of the control apparatus 1-2 according to the embodiment.

FIG. 11 is a block diagram illustrating an example of a functional configuration of a control apparatus 1-3 according to a third embodiment of the present disclosure.

FIG. 12 is a flowchart illustrating an exemplary operation of the control apparatus 1-3 according to the embodiment.

FIG. 13 is a block diagram illustrating an example of a functional configuration of a control apparatus 1-4 according to a fourth embodiment of the present disclosure.

FIG. 14 illustrates an initial state in which an affected area (observation target) can be observed.

FIG. 15 is a plan view in a state where the position and the attitude of a camera have been changed.

FIG. 16 is an explanatory diagram for describing a procedure for calculating a suitable angle in more detail.

FIG. 17 includes explanatory diagrams illustrating an example of navigation information according to the embodiment.

FIG. 18 is a flowchart illustrating an exemplary operation of the control apparatus 1-4 according to the embodiment.

FIG. 19 is a block diagram illustrating an example of a functional configuration of a control apparatus 1-5 according to a fifth embodiment of the present disclosure.

FIG. 20 is a flowchart illustrating an exemplary operation of the control apparatus 1-5 according to the embodiment.

FIG. 21 is an explanatory diagram illustrating a hardware configuration example.

### MODE(S) FOR CARRYING OUT THE INVENTION

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

Note that description will be provided in the following order.

### <<1. Overview>>

### <<2. Embodiments>>

<2-1. First embodiment>
<2-2. Second embodiment>
<2-3. Third embodiment>
<2-4. Fourth embodiment>
<2-5. Fifth embodiment>
<<3. Hardware configuration example>>

### <<4. Conclusion>>

### 1. OVERVIEW

First, in order to render the present disclosure clearer, a medical observation system to which embodiments according to the present disclosure may be applied will be described.

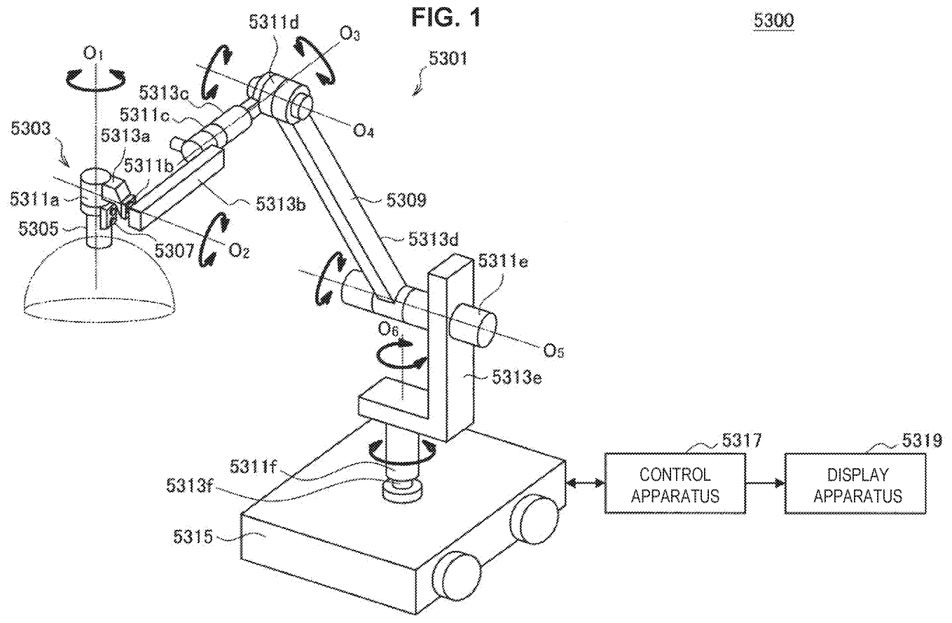

FIG. 1 is a view depicting an example of a schematic configuration of a medical observation system 5300 to which the embodiment according to an embodiment of the present disclosure can be applied. Referring to FIG. 1, the medical observation system 5300 includes a microscope apparatus 5301, a control apparatus 5317 and a display apparatus 5319. It is to be noted that, in the description of the medical observation system 5300, the term “user” signifies an arbitrary one of medical staff members such as a surgery or an assistant who uses the medical observation system 5300.

The microscope apparatus 5301 has a microscope unit 5303 (image pickup apparatus) for enlarging an observation target (surgical region of a patient, or the like) for observation, an arm unit 5309 which supports the microscope unit 5303 at a distal end thereof, and a base unit 5315 which supports a proximal end of the arm unit 5309.

The microscope unit 5303 includes a cylindrical portion 5305 of a substantially cylindrical shape, an image pickup unit (not depicted) provided in the inside of the cylindrical portion 5305, and an operation unit 5307 provided in a partial region of an outer circumference of the cylindrical portion 5305. The microscope unit 5303 is a microscope unit of the electronic image pickup type (microscope unit of the video type) which picks up an image electronically by the image pickup unit.

A cover glass member for protecting the internal image pickup unit is provided at an opening face of a lower end of the cylindrical portion 5305. Light from an observation target (hereinafter referred to also as observation light) passes through the cover glass member and enters the image pickup unit in the inside of the cylindrical portion 5305. It is to be noted that a light source includes, for example, a light emitting diode (LED) or the like may be provided in the inside of the cylindrical portion 5305, and upon image picking up, light may be irradiated upon an observation target from the light source through the cover glass member.

The image pickup unit includes an optical system which condenses observation light, and an image pickup element which receives the observation light condensed by the optical system. The optical system includes a combination of a plurality of lenses including a zoom lens and a focusing lens. The optical system has optical properties adjusted such that the observation light is condensed to be formed image on a light receiving face of the image pickup element. The image pickup element receives and photoelectrically converts the observation light to generate a signal corresponding to the observation light, namely, an image signal corresponding to an observation image. As the image pickup element, for example, an image pickup element which has a Bayer array and is capable of picking up an image in color is used. The image pickup element may be any of various known image pickup elements such as a complementary metal oxide semiconductor (CMOS) image sensor or a charge coupled device (CCD) image sensor. The image signal generated by the image pickup element is transmitted as RAW data to the control apparatus 5317. Here, the transmission of the image signal may be performed suitably by optical communication. This is because, since, at a surgery site, the surgeon performs surgery while observing the state of an affected area through a picked up image, in order to achieve surgery with a higher degree of safety and certainty, it is demanded for a moving image of the surgical region to be displayed on the real time basis as far as possible. Where optical communication is used to transmit the image signal, the picked up image can be displayed with low latency.

It is to be noted that the image pickup unit may have a driving mechanism for moving the zoom lens and the focusing lens of the optical system thereof along the optical axis. Where the zoom lens and the focusing lens are moved suitably by the driving mechanism, the magnification of the picked up image and a focusing distance (distance to a focused observation target) upon image picking up can be adjusted. Further, the image pickup unit may incorporate therein various functions which may be provided generally in a microscopic unit of the electronic image pickup such as an auto exposure (AE) function or an auto focus (AF) function.

Further, the image pickup unit may be configured as an image pickup unit of the single-plate type which includes a single image pickup element or may be configured as an image pickup unit of the multi-plate type which includes a plurality of image pickup elements. Where the image pickup unit is configured as that of the multi-plate type, for example, image signals corresponding to red, green, and blue colors may be generated by the image pickup elements and may be synthesized to obtain a color image. Alternatively, the image pickup unit may be configured such that it has a pair of image pickup elements for acquiring image signals for the right eye and the left eye compatible with a stereoscopic vision (three dimensional (3D) display). Where 3D display is applied, the surgeon can comprehend the depth of a living body tissue in the surgical region with a higher degree of accuracy. It is to be noted that, if the image pickup unit is configured as that of stereoscopic type, then a plurality of optical systems is provided corresponding to the individual image pickup elements.

The operation unit 5307 includes, for example, a cross lever, a switch or the like and accepts an operation input of the user. For example, the user can input an instruction to change the magnification of the observation image and the focusing distance to the observation target through the operation unit 5307. The magnification and the focusing distance can be adjusted by the driving mechanism of the image pickup unit suitably moving the zoom lens and the focusing lens in accordance with the instruction. Further, for example, the user can input an instruction to switch the operation mode of the arm unit 5309 (an all-free mode and a fixed mode hereinafter described) through the operation unit 5307. It is to be noted that when the user intends to move the microscope unit 5303, it is supposed that the user moves the microscope unit 5303 in a state in which the user grasps the microscope unit 5303 holding the cylindrical portion 5305. Accordingly, the operation unit 5307 is preferably provided at a position at which it can be operated readily by the fingers of the user with the cylindrical portion 5305 held such that the operation unit 5307 can be operated even while the user is moving the cylindrical portion 5305.

The arm unit 5309 is configured such that a plurality of links (first link 5313a to sixth link 5313f) is connected for rotation relative to each other by a plurality of joint portions (first joint portion 5311a to sixth joint portion 5311f).

The first joint portion 5311a has a substantially columnar shape and supports, at a distal end (lower end) thereof, an upper end of the cylindrical portion 5305 of the microscope unit 5303 for rotation around an axis of rotation (first axis O₁) parallel to the center axis of the cylindrical portion 5305. Here, the first joint portion 5311a may be configured such that the first axis O₁ thereof is in alignment with the optical axis of the image pickup unit of the microscope unit 5303. By the configuration, if the microscope unit 5303 is rotated around the first axis O₁, then the field of view can be changed so as to rotate the picked up image.

The first link 5313a fixedly supports, at a distal end thereof, the first joint portion 5311a. Specifically, the first link 5313a is a bar-like member having a substantially L shape and is connected to the first joint portion 5311a such that one side at the distal end side thereof extends in a direction orthogonal to the first axis O₁ and an end portion of the one side abuts with an upper end portion of an outer periphery of the first joint portion 5311a. The second joint portion 5311b is connected to an end portion of the other side on the proximal end side of the substantially L shape of the first link 5313a.

The second joint portion 5311b has a substantially columnar shape and supports, at a distal end thereof, a proximal end of the first link 5313a for rotation around an axis of rotation (second axis O₂) orthogonal to the first axis O₁. The second link 5313b is fixedly connected at a distal end thereof to a proximal end of the second joint portion 5311b.

The second link 5313b is a bar-like member having a substantially L shape, and one side of a distal end side of the second link 5313b extends in a direction orthogonal to the second axis O₂ and an end portion of the one side is fixedly connected to a proximal end of the second joint portion 5311b. The third joint portion 5311c is connected to the other side at the proximal end side of the substantially L shape of the second link 5313b.

The third joint portion 5311c has a substantially columnar shape and supports, at a distal end thereof, a proximal end of the second link 5313b for rotation around an axis of rotation (third axis O₃) orthogonal to the first axis O₁ and the second axis O₂. The third link 5313c is fixedly connected at a distal end thereof to a proximal end of the third joint portion 5311c. By rotating the components at the distal end side including the microscope unit 5303 around the second axis O₂ and the third axis O₃, the microscope unit 5303 can be moved such that the position of the microscope unit 5303 is changed within a horizontal plane. In other words, by controlling the rotation around the second axis O₂ and the third axis O₃, the field of view of the picked up image can be moved within a plane.

The third link 5313c is configured such that the distal end side thereof has a substantially columnar shape, and a proximal end of the third joint portion 5311c is fixedly connected to the distal end of the columnar shape such that both of them have a substantially same center axis. The proximal end side of the third link 5313c has a prismatic shape, and the fourth joint portion 5311d is connected to an end portion of the third link 5313c.

The fourth joint portion 5311d has a substantially columnar shape and supports, at a distal end thereof, a proximal end of the third link 5313c for rotation around an axis of rotation (fourth axis O₄) orthogonal to the third axis O₃. The fourth link 5313d is fixedly connected at a distal end thereof to a proximal end of the fourth joint portion 5311d.

The fourth link 5313d is a bar-like member extending substantially linearly and is fixedly connected to the fourth joint portion 5311d such that it extends orthogonally to the fourth axis O₄ and abuts at an end portion of the distal end thereof with a side face of the substantially columnar shape of the fourth joint portion 5311d. The fifth joint portion 5311e is connected to a proximal end of the fourth link 5313d.

The fifth joint portion 5311e has a substantially columnar shape and supports, at a distal end side thereof, a proximal end of the fourth link 5313d for rotation around an axis of rotation (fifth axis O₅) parallel to the fourth axis O₄. The fifth link 5313e is fixedly connected at a distal end thereof to a proximal end of the fifth joint portion 5311e. The fourth axis O₄ and the fifth axis O₅ are axes of rotation around which the microscope unit 5303 can be moved in the upward and downward direction. By rotating the components at the distal end side including the microscope unit 5303 around the fourth axis O₄ and the fifth axis O₅, the height of the microscope unit 5303, namely, the distance between the microscope unit 5303 and an observation target, can be adjusted.

The fifth link 5313e includes a combination of a first member having a substantially L shape one side of which extends in the vertical direction and the other side of which extends in the horizontal direction, and a bar-like second member extending vertically downwardly from the portion of the first member which extends in the horizontal direction. The fifth joint portion 5311e is fixedly connected at a proximal end thereof to a neighboring upper end of a part extending the first member of the fifth link 5313e in the vertical direction. The sixth joint portion 5311f is connected to proximal end (lower end) of the second member of the fifth link 5313e.

The sixth joint portion 5311f has a substantially columnar shape and supports, at a distal end side thereof, a proximal end of the fifth link 5313e for rotation around an axis of rotation (sixth axis O₆) parallel to the vertical direction. The sixth link 5313f is fixedly connected at a distal end thereof to a proximal end of the sixth joint portion 5311f.

The sixth link 5313f is a bar-like member extending in the vertical direction and is fixedly connected at a proximal end thereof to an upper face of the base unit 5315.

The first joint portion 5311a to sixth joint portion 5311f have movable ranges suitably set such that the microscope unit 5303 can make a desired movement. Consequently, in the arm unit 5309 having the configuration described above, a movement of totaling six degrees of freedom including three degrees of freedom for translation and three degrees of freedom for rotation can be implemented with regard to a movement of the microscope unit 5303. By configuring the arm unit 5309 such that six degrees of freedom are implemented for movements of the microscope unit 5303 in this manner, the position and the attitude of the microscope unit 5303 can be controlled freely within the movable range of the arm unit 5309. Accordingly, it is possible to observe a surgical region from every angle, and surgery can be executed more smoothly.

It is to be noted that the configuration of the arm unit 5309 as depicted is an example at all, and the number and shape (length) of the links including the arm unit 5309 and the number, location, direction of the axis of rotation and so forth of the joint portions may be designed suitably such that desired degrees of freedom can be implemented. For example, in order to freely move the microscope unit 5303, preferably the arm unit 5309 is configured so as to have six degrees of freedom as described above. However the arm unit 5309 may also be configured so as to have much greater degree of freedom (namely, redundant degree of freedom). Where a redundant degree of freedom exists, it is possible to change the attitude of the arm unit 5309 in a state in which the position and the attitude of the microscope unit 5303 are fixed. Accordingly, control can be implemented which is higher in convenience to the surgeon such as to control the attitude of the arm unit 5309 such that, for example, the arm unit 5309 does not interfere with the field of view of the surgeon who watches the display apparatus 5319.

Here, an actuator in which a driving mechanism such as a motor, an encoder which detects an angle of rotation at each joint portion and so forth are incorporated may be provided for each of the first joint portion 5311a to sixth joint portion 5311f. By suitably controlling driving of the actuators provided in the first joint portion 5311a to sixth joint portion 5311f by the control apparatus 5317, the attitude of the arm unit 5309, namely, the position and the attitude of the microscope unit 5303, can be controlled. Specifically, the control apparatus 5317 can comprehend the attitude of the arm unit 5309 at present and the position and the attitude of the microscope unit 5303 at present on the basis of information regarding the angle of rotation of the joint portions detected by the encoders. The control apparatus 5317 uses the comprehended information to calculate a control value (for example, an angle of rotation or torque to be generated) for each joint portion with which a movement of the microscope unit 5303 in accordance with an operation input from the user is implemented. Accordingly the control apparatus 5317 drives driving mechanism of the each joint portion in accordance with the control value. It is to be noted that, in this case, the control method of the arm unit 5309 by the control apparatus 5317 is not limited, and various known control methods such as force control or position control may be applied.

For example, when the surgeon performs operation inputting suitably through an inputting apparatus not depicted, driving of the arm unit 5309 may be controlled suitably in response to the operation input by the control apparatus 5317 to control the position and the attitude of the microscope unit 5303. By this control, it is possible to support, after the microscope unit 5303 is moved from an arbitrary position to a different arbitrary position, the microscope unit 5303 fixedly at the position after the movement.

Further, where force control is applied, the control apparatus 5317 may perform power-assisted control to drive the actuators of the first joint portion 5311a to sixth joint portion 5311f such that the arm unit 5309 may receive external force by the user and move smoothly following the external force. This makes it possible to move, when the user holds and directly moves the position of the microscope unit 5303, the microscope unit 5303 with comparatively weak force. Accordingly, it becomes possible for the user to move the microscope unit 5303 more intuitively by a simpler and easier operation, and the convenience to the user can be improved.

Further, driving of the arm unit 5309 may be controlled such that the arm unit 5309 performs a pivot movement. The pivot movement here is a motion for moving the microscope unit 5303 such that the direction of the optical axis of the microscope unit 5303 is kept toward a predetermined point (hereinafter referred to as pivot point) in a space. Since the pivot movement makes it possible to observe the same observation position from various directions, more detailed observation of an affected area becomes possible. It is to be noted that, where the microscope unit 5303 is configured such that the focusing distance thereof is fixed, preferably the pivot movement is performed in a state in which the distance between the microscope unit 5303 and the pivot point is fixed. In this case, it is sufficient if the distance between the microscope unit 5303 and the pivot point is adjusted to a fixed focusing distance of the microscope unit 5303 in advance. By the configuration just described, the microscope unit 5303 comes to move on a hemispherical plane (schematically depicted in FIG. 1) having a diameter corresponding to the focusing distance centered at the pivot point, and even if the observation direction is changed, a clear picked up image can be obtained. On the other hand, where the microscope unit 5303 is configured such that the focusing distance thereof is adjustable, the pivot movement may be performed in a state in which the distance between the microscope unit 5303 and the pivot point is variable. In this case, for example, the control apparatus 5317 may calculate the distance between the microscope unit 5303 and the pivot point on the basis of information regarding the angles of rotation of the joint portions detected by the encoders and automatically adjust the focusing distance of the microscope unit 5303 on the basis of a result of the calculation. Alternatively, where the microscope unit 5303 includes an AF function, adjustment of the focusing distance may be performed automatically by the AF function every time the changing in distance caused by the pivot movement between the microscope unit 5303 and the pivot point.

Further, each of the first joint portion 5311a to sixth joint portion 5311f may be provided with a brake for constraining the rotation of the first joint portion 5311a to sixth joint portion 5311f. Operation of the brake may be controlled by the control apparatus 5317. For example, if it is intended to fix the position and the attitude of the microscope unit 5303, then the control apparatus 5317 renders the brakes of the joint portions operative. Consequently, even if the actuators are not driven, the attitude of the arm unit 5309, namely, the position and the attitude of the microscope unit 5303, can be fixed, and therefore, the power consumption can be reduced. When it is intended to move the position and the attitude of the microscope unit 5303, it is sufficient if the control apparatus 5317 releases the brakes of the joint portions and drives the actuators in accordance with a predetermined control method.

Such operation of the brakes may be performed in response to an operation input by the user through the operation unit 5307 described hereinabove. In a case where the user intends to move the position and the attitude of the microscope unit 5303, the user would operate the operation unit 5307 to release the brakes of the joint portions. Consequently, the operation mode of the arm unit 5309 changes to a mode in which rotation of the joint portions can be performed freely (all-free mode). On the other hand, in a case where the user intends to fix the position and the attitude of the microscope unit 5303, then the user would operate the operation unit 5307 to render the brakes of the joint portions operative. Consequently, the operation mode of the arm unit 5309 changes to a mode in which rotation of the joint portions is constrained (fixed mode).

The control apparatus 5317 integrally controls operation of the medical observation system 5300 by controlling operation of the microscope apparatus 5301 and the display apparatus 5319. For example, the control apparatus 5317 renders the actuators of the first joint portion 5311a to sixth joint portion 5311f operative in accordance with a predetermined control method to control driving of the arm unit 5309 (perform arm control processing). Further, for example, the control apparatus 5317 controls operation of the brakes of the first joint portion 5311a to sixth joint portion 5311f to change the operation mode of the arm unit 5309. Further, for example, the control apparatus 5317 performs various signal processes for an image signal (image processing) acquired by the image pickup unit of the microscope unit 5303 of the microscope apparatus 5301 to generate image data for display and controls the display apparatus 5319 to display the generated image data. As the signal processes, various known signal processes such as, for example, a development process (demosaic process), an image quality improving process (a bandwidth enhancement process, a super-resolution process, a noise reduction (NR) process and/or an image stabilization process) and/or an enlargement process (namely, an electronic zooming process) may be performed.

It is to be noted that communication between the control apparatus 5317 and the microscope unit 5303 and communication between the control apparatus 5317 and the first joint portion 5311a to sixth joint portion 5311f may be wired communication or wireless communication. Where wired communication is applied, communication by an electric signal may be performed or optical communication may be performed. In this case, a cable for transmission used for wired communication may be configured as an electric signal cable, an optical fiber or a composite cable of them in response to an applied communication method. On the other hand, where wireless communication is applied, since there is no necessity to lay a transmission cable in the operating room, such a situation that movement of medical staff in the operating room is disturbed by a transmission cable can be eliminated.

The control apparatus 5317 may be a processor such as a central processing unit (CPU) or a graphics processing unit (GPU), or a microcomputer or a control board in which a processor and a storage element such as a memory are incorporated. The various functions described hereinabove can be implemented by the processor of the control apparatus 5317 operating in accordance with a predetermined program. It is to be noted that, in the example depicted, the control apparatus 5317 is provided as an apparatus separate from the microscope apparatus 5301. However, the control apparatus 5317 may be installed in the inside of the base unit 5315 of the microscope apparatus 5301 and configured integrally with the microscope apparatus 5301. The control apparatus 5317 may also include a plurality of apparatus. For example, microcomputers, control boards or the like may be disposed in the microscope unit 5303 and the first joint portion 5311a to sixth joint portion 5311f of the arm unit 5309 and connected for communication with each other to implement functions similar to those of the control apparatus 5317.

The display apparatus 5319 is provided in the operating room and displays an image corresponding to image data generated by the control apparatus 5317 under the control of the control apparatus 5317. In other words, an image of a surgical region picked up by the microscope unit 5303 is displayed on the display apparatus 5319. The display apparatus 5319 may display, in place of or in addition to an image of a surgical region, various kinds of information relating to the surgery such as physical information of a patient or information regarding a surgical procedure of the surgery. In this case, the display of the display apparatus 5319 may be switched suitably in response to an operation by the user. Alternatively, a plurality of such display apparatus 5319 may also be provided such that an image of a surgical region or various kinds of information relating to the surgery may individually be displayed on the plurality of display apparatus 5319. It is to be noted that, as the display apparatus 5319, various known display apparatus such as a liquid crystal display apparatus or an electro luminescence (EL) display apparatus may be applied.

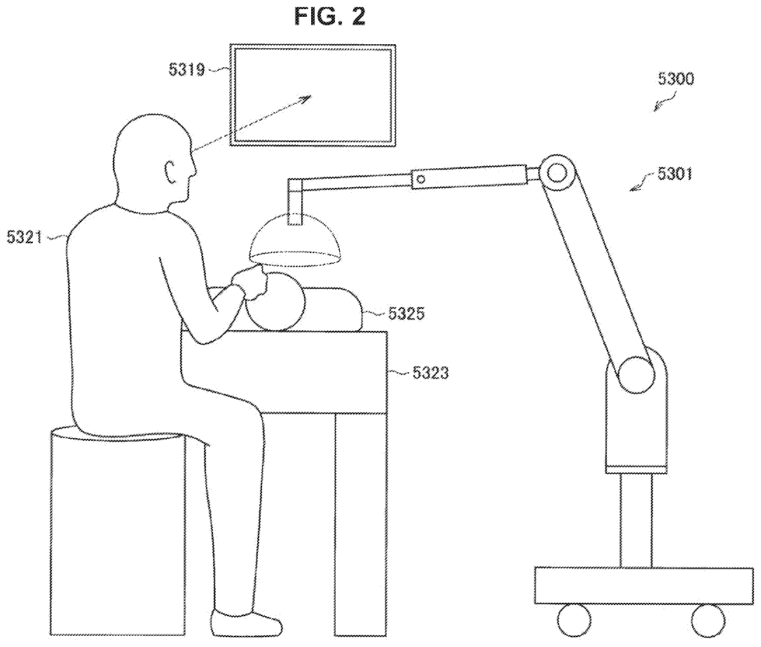

FIG. 2 is a view illustrating a state of surgery in which the medical observation system 5300 depicted in FIG. 1 is used. FIG. 2 schematically illustrates a state in which a surgeon 5321 uses the medical observation system 5300 to perform surgery for a patient 5325 on a patient bed 5323. It is to be noted that, in FIG. 2, for simplified illustration, the control apparatus 5317 from among the components of the medical observation system 5300 is omitted and the microscope apparatus 5301 is depicted in a simplified from.

As depicted in FIG. 2, upon surgery, using the medical observation system 5300, an image of a surgical region picked up by the microscope apparatus 5301 is displayed in an enlarged scale on the display apparatus 5319 installed on a wall face of the operating room. The display apparatus 5319 is installed at a position opposing to the surgeon 5321, and the surgeon 5321 would perform various treatments for the surgical region such as, for example, resection of the affected area while observing a state of the surgical region from a video displayed on the display apparatus 5319.

An example of the medical observation system 5300 to which embodiments according to the present disclosure may be applied has been described above. Subsequently, a case where the medical observation system 5300 described above is used for a dental treatment will be studied.

In a dental treatment, while observing a mirror reflection image (affected area) of a dental mirror grasped by one hand, a user performs a treatment using a surgical instrument grasped by the other hand in some cases. Consequently, the both hands of a surgeon during a dental treatment are occupied by the dental mirror and the surgical instrument, so that it may be difficult to move the arm unit 5309 described above. As a result, it is difficult in some cases for the user to obtain a desired field of view for freely observing an affected area (observation target).

Further, in a case where an optical microscope is used, the line of sight of the user and the optical axis of the optical microscope agree, and thus, it is possible for the user to perform angle adjustment and position adjustment for the dental mirror in a sense similar to that in a case of using the dental mirror with the naked eyes. Consequently, in the case where an optical microscope is used, it is possible for the user to perform angle adjustment and position adjustment for the dental mirror intuitively, and discomfort is less likely to occur particularly for a user who is familiar with a dental treatment by means of a dental mirror with the naked eyes.

On the other hand, in a case where the medical observation system 5300 having a video microscope (the microscope unit 5303) is used, the line of sight of the user (the surgeon 5321) is directed to the display apparatus 5319 as illustrated in FIG. 2. In such a case, since the line of sight of the user and the optical axis of the microscope unit 5303 do not agree, the user will perform angle adjustment and position adjustment for the dental mirror while being aware of the position and the angle with respect to the optical axis of the microscope unit 5303 rather than his/her own line of sight. Consequently, it is not easy for the user to perform angle adjustment and position adjustment for the dental mirror, and it is difficult to freely observe an affected area (observation target).

Therefore, using the above-described circumstance as a viewpoint, embodiments according to the present disclosure have been devised. In accordance with the embodiments according to the present disclosure, it is possible to freely observe an affected area (observation target) by performing image processing on a picked up image or arm control processing of controlling the arm unit 5309 on the basis of dental mirror detection information obtained from the picked up image. Hereinafter, some embodiments of the present disclosure that achieve the above-described effects will be described.

### 2. EMBODIMENTS

### 2-1. First Embodiment

In a case where a user observes an affected area using a video microscope and a dental mirror, when the proportion that a mirror portion at the leading end of the dental mirror occupies in an output image displayed on the display apparatus is small, it is difficult for the user to comfortably observe the affected area reflected on the mirror portion as a mirror reflection image. Therefore, as a first embodiment according to the present disclosure, a control apparatus that enables an output image in which the proportion that the mirror portion occupies is large to be obtained will be described.

### (Exemplary Configuration)

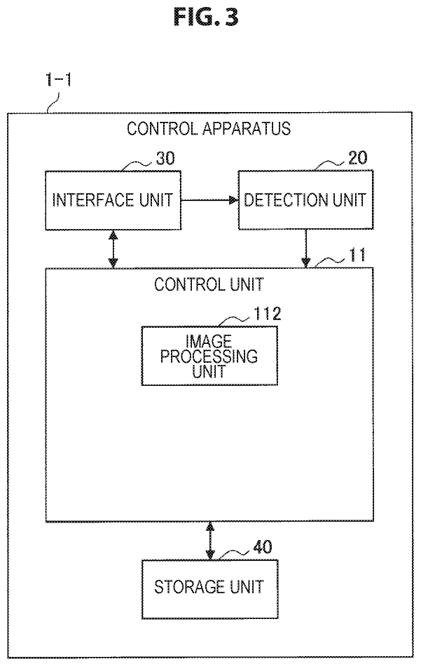

First, an exemplary configuration of the first embodiment of the present disclosure will be described with reference to FIG. 3. FIG. 3 is a block diagram illustrating an example of a functional configuration of a control apparatus 1-1 according to the present embodiment. The control apparatus 1-1 illustrated in FIG. 3 is equivalent to the control apparatus 5317 described with reference to FIG. 1. As illustrated in FIG. 3, the control apparatus 1-1 includes a control unit 11, a detection unit 20, an interface unit 30, and a storage unit 40.

The control unit 11 controls each component of the control apparatus 1-1. Further, the control unit 11 according to the present embodiment also functions as an image processing unit 112. Hereinafter, after describing the overall configuration of the control apparatus 1-1, the functions of the control unit 11 as the image processing unit 112 will be described.

The detection unit 20 functions as an acquisition unit that detects a dental mirror from a picked up image acquired by the microscope unit 5303 described with reference to FIG. 1, and acquires dental mirror detection information related to detection of the dental mirror.

For example, the detection unit 20 may detect the mirror portion at the leading end of the dental mirror that reflects a mirror reflection image, of the dental mirror. Further, the dental mirror detection information acquired by the detection unit 20 and provided for the control unit 11 may include information indicating the position of the mirror portion (for example, the center of gravity of the mirror portion), information indicating a region of the mirror portion, and information indicating whether or not detection of the mirror portion has succeeded. It is to be noted that detection of the mirror portion performed by the detection unit 20 may be performed by a well-known object detection technology.

The interface unit 30 is an interface for input/output. For example, the interface unit 30 performs input/output of information between the microscope apparatus 5301 and the display apparatus 5319 described with reference to FIG. 1. For example, the control unit 11 may acquire a picked up image obtained by the microscope unit 5303 from the microscope apparatus 5301 via the interface unit 30. Further, the control unit 11 may cause the microscope apparatus 5301 to output a control signal for controlling the arm unit 5309 via the interface unit 30. Further, the control unit 11 may cause an output image to be output to the display apparatus 5319 via the interface unit 30.

The storage unit 40 stores programs and parameters for the respective components of the control apparatus 1-1 to function.

The overall configuration of the control apparatus 1-1 has been described above. Subsequently, the functions of the control unit 11 as the image processing unit 112 will be described.

The image processing unit 112 performs image processing on a picked up image on the basis of the dental mirror detection information provided from the detection unit 20. The image processing unit 112 according to the present embodiment may perform image processing of specifying a cutout region on the basis of the dental mirror detection information, for example, and cutting the cutout region out of the picked up image.

For example, in a case where the picked up image has a so-called 4K resolution, and the display apparatus 5319 can make display in the 2K resolution, for example, the image processing unit 112 may specify a region equivalent to the 2K resolution including the region of the mirror portion as the cutout region. Further, the image processing unit 112 may cause the cutout region cut out of the picked up image to be output to the display apparatus 5319 as an output image.

Further, the image processing unit 112 may specify a region that includes the region of the mirror portion and that is smaller than a region equivalent to the resolution of the display apparatus 5319 as the cutout region. In such a case, the image processing unit 112 may perform enlarging processing after cutting out the cutout region to generate an output image to be output to the display apparatus 5319. It is to be noted that image enlarging processing performed by the image processing unit 112 may include super-resolution processing, or may include well-known image enlarging processing such as the Bi-cubic method or Bi-linear method.

It is to be noted that the image processing unit 112 may specify the size of the cutout region on the basis of the area of the region of the detected mirror portion, or on the basis of a user input via a foot switch not illustrated or the like. Further, the image processing unit 112 may specify the cutout region such that the center position of the cutout region is brought to the position of the mirror portion.

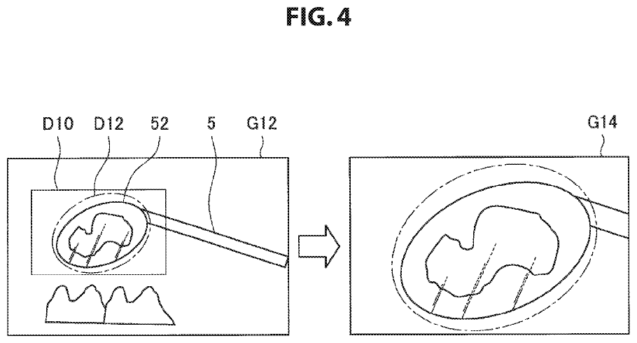

FIG. 4 includes explanatory diagrams illustrating an example of cutting out processing and image enlarging processing performed by the image processing unit 112. For example, the mirror portion 52 at the leading end of a dental mirror 5 is detected by the detection unit 20 from a picked up image G12 illustrated in FIG. 4. For example, the detection unit 20 may provide a region D12 for the control unit 11 as a detected region of the mirror portion 52. The image processing unit 112 of the control unit 11 specifies such a cutout region D10 that includes the region D12, and after cutting out the cutout region D10, performs image enlarging processing to generate an output image G14 to be displayed on the display apparatus 5319.

With such a configuration, even in a case where the proportion that the region of the mirror portion at the leading end of the dental mirror occupies in the picked up image is small, the mirror portion can be displayed on the display apparatus 5319 in a sufficient size, so that is possible for the user to comfortably observe the affected area.

### (Exemplary Operation)

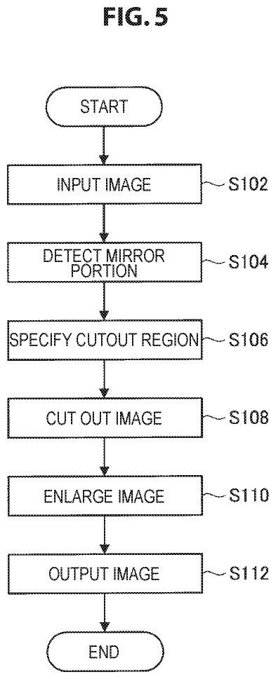

An exemplary configuration of the control apparatus 1-1 according to the present embodiment has been described above. Subsequently, an exemplary operation of the present embodiment will be described with reference to FIG. 5. FIG. 5 is a flowchart illustrating an exemplary operation of the control apparatus 1-1 according to the present embodiment.

As illustrated in FIG. 5, first, a picked up image is input via the interface unit 30 (S102). Subsequently, the mirror portion at the leading end of the dental mirror is detected by the detection unit 20 (S104).

Subsequently, the image processing unit 112 specifies a cutout region on the basis of the region of the mirror portion provided by the detection unit 20 (S106), and cuts the cutout region out of the picked up image (S108).

Furthermore, the image processing unit 112 enlarges the cutout region obtained in step S108 according to necessity (S110), and causes the obtained output image to be output to the display apparatus 5319 (S112).

The processing in steps S102 to S112 described above may be repeated as appropriate.

### (Effects)

The first embodiment of the present disclosure has been described above. In accordance with the first embodiment of the present disclosure, even in a case where the proportion that the region of the mirror portion occupies in the picked up image is small, the mirror portion that reflects a mirror reflection image can be displayed in a sufficient size, and it is possible for the user to comfortably observe the affected area.

It is to be noted that, since the control apparatus 1-1 according to the present embodiment may not perform the arm control processing, the present embodiment can be applied even in a case where the microscope apparatus 5301 illustrated in FIG. 1 does not include the driving mechanism of the arm unit 5309.

### 2-2. Second Embodiment

In the above-described first embodiment, an example of obtaining a user desired output image (an image in which the proportion that the mirror portion occupies is larger) through image processing has been described. Subsequently, as a second embodiment of the present disclosure, an example of performing arm control processing in addition to the image processing will be described.

### (Exemplary Configuration)

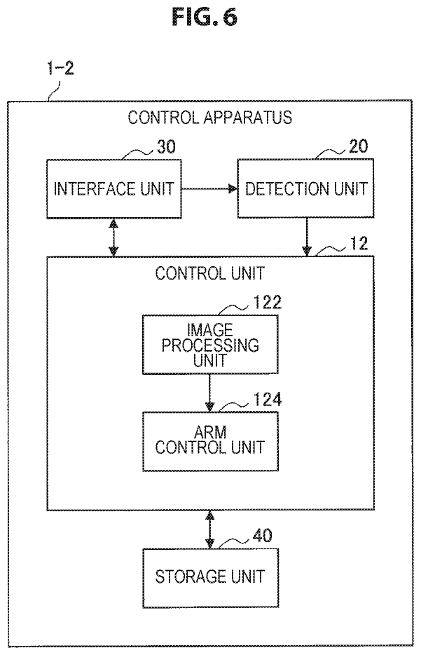

First, an exemplary configuration of the second embodiment of the present disclosure will be described with reference to FIG. 6. FIG. 6 is a block diagram illustrating an example of a functional configuration of a control apparatus 1-2 according to the second embodiment of the present disclosure. The control apparatus 1-2 illustrated in FIG. 6 is equivalent to the control apparatus 5317 described with reference to FIG. 1. As illustrated in FIG. 6, the control apparatus 1-1 includes a control unit 12, the detection unit 20, the interface unit 30, and the storage unit 40. In the components illustrated in FIG. 6, the components of the detection unit 20, the interface unit 30, and the storage unit 40 are substantially identical to the components of the detection unit 20, the interface unit 30, and the storage unit 40 described with reference to FIG. 3, respectively, and description here is thus omitted.

The control unit 12 controls each component of the control apparatus 1-2. Further, the control unit 12 according to the present embodiment also functions as an image processing unit 122 and an arm control unit 124 as illustrated in FIG. 6.

The image processing unit 122 performs image processing on a picked up image on the basis of the dental mirror detection information provided from the detection unit 20, similarly to the image processing unit 112 described with reference to FIG. 3. For example, the image processing unit 122 may perform cutout region specifying processing, cutting out processing, enlarging processing, and the like, similarly to the image processing unit 112.

Further, the image processing unit 122 according to the present embodiment may determine whether or not the cutout region is about to fall outside the image pickup range when specifying the cutout region. For example, the image processing unit 122 may determine whether or not the cutout region is about to fall outside the image pickup range by determining whether or not the cutout region is included in a predetermined outer peripheral region in the picked up image. Further, the image processing unit 122 may determine whether or not the cutout region is about to fall outside the image pickup range by determining whether or not at least part of the region of the mirror portion used for specifying the cutout region is included in the predetermined outer peripheral region in the picked up image. The image processing unit 122 provides a result of the determination for the arm control unit 124. With such a configuration, in a case where the cutout region is about to fall outside the image pickup range, the arm control unit 124 which will be described later can perform arm control processing to prevent the mirror portion from falling outside the image pickup range.

The arm control unit 124 performs arm control processing of controlling the arm unit 5309 described with reference to FIG. 1 and FIG. 2. For example, the arm control unit 124 according to the present embodiment may perform the arm control processing on the basis of the position of the detected mirror portion.

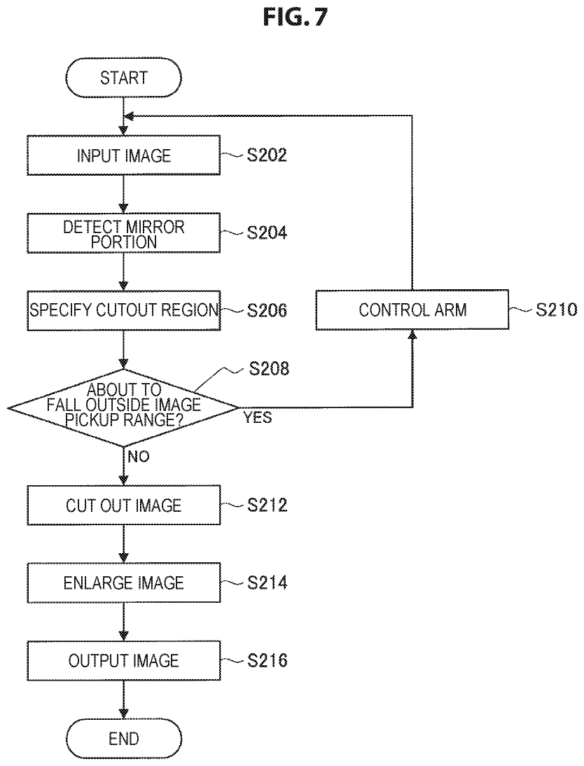

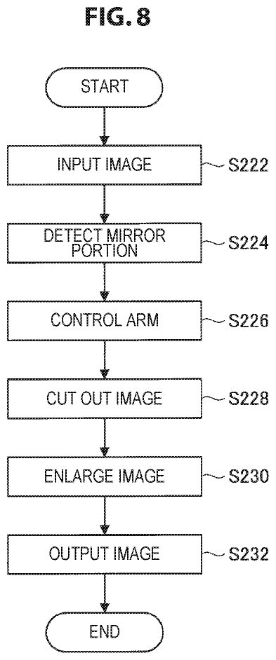

The arm control unit 124 may perform the arm control processing such that the position of the mirror portion, for example, is brought to a predetermined position (for example, the center position) in the picked up image. The arm control processing performed by the arm control unit 124 may be performed in a case where it is determined by the image processing unit 122 that the cutout region is about to fall outside the image pickup range, for example. An exemplary operation in such a case will be described later with reference to FIG. 7. Further, the arm control processing performed by the arm control unit 124 may be performed all the time. An exemplary operation in such a case will be described later with reference to FIG. 8.

With such a configuration, it is possible to prevent the mirror portion from falling outside the image pickup range. Consequently, in a case where a surgical field is wider than an image pickup range initially set by the user, it is possible to save the user the effort of setting the image pickup range again manually, for example.

Further, the arm control unit 124 may perform the arm control processing such that the region of the detected mirror portion is included in the picked up image, and the proportion of the region of the mirror portion occupies in the picked up image becomes larger. It is to be noted that such arm control processing may be achieved by visual feedback control, for example. In accordance with such arm control processing, it is possible to obtain, by image pickup, an image having an angle of view similar to that of an image obtained by the cutting out processing performed by the image processing unit 122. Accordingly, the cutting out processing and the enlarging processing performed by the image processing unit 122 may not be performed in such a case, and an output image may have a higher image quality than in the case where the cutting out processing and the enlarging processing are performed. In such an example, the arm control processing is performed such that the image pickup range of the microscope unit 5303 tracks the mirror portion, and thus, such arm control processing may be hereinafter called tracking of the mirror portion.

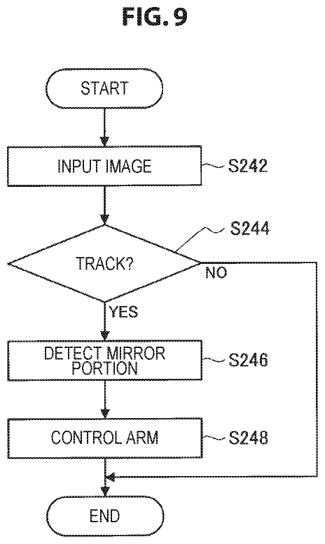

It is to be noted that tracking of the mirror portion may be performed all the time in accordance with previously-set mode setting of the medical observation system 5300, for example. Further, the mode may be switched on the basis of a user input via a foot switch not illustrated or the like. An exemplary operation in such a case will be described later with reference to FIG. 9.

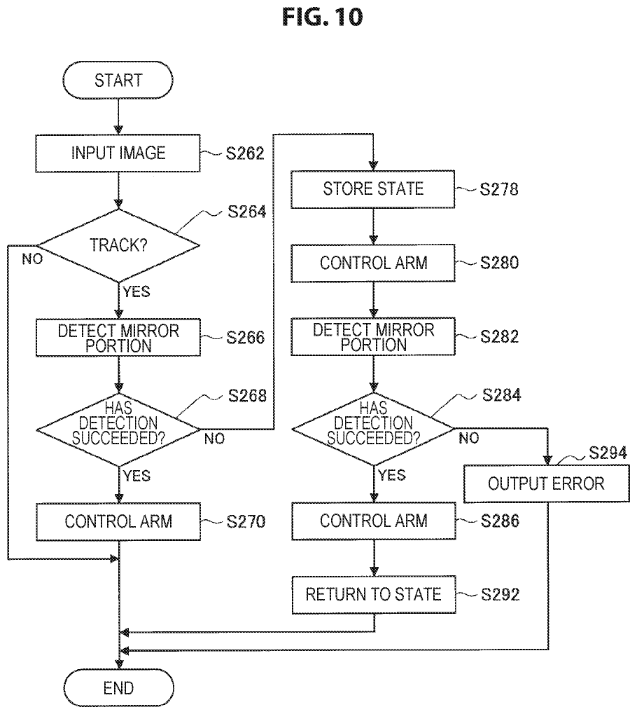

Further, tracking of the mirror portion may be performed only at a timing instructed by a user input via a foot switch not illustrated or the like. In such a case, the mirror portion is not tracked continuously, so that the detection unit 20 may fail in detection of the mirror portion from the picked up image. Therefore, the arm control unit 124 may perform the arm control processing such that the image pickup range of the microscope unit 5303 is widened in a case where information indicating that detection of the mirror portion has failed is included in the dental mirror detection information. For example, the arm control unit 124 may perform the arm control processing such that the microscope unit 5303 is moved backward. An exemplary operation in such a case will be described later with reference to FIG. 10.

With such a configuration, the arm control processing is performed such that the image pickup range of the microscope unit 5303 is widened even in a case where the mirror portion is not included in the picked up image and the detection unit 20 has failed in detection of the mirror portion, so that the detection unit 20 may detect the mirror portion.

### (Exemplary Operation)

An exemplary configuration of the control apparatus 1-2 according to the present embodiment has been described above. Subsequently, some exemplary operations according to the present embodiment will be described with reference to FIG. 7 to FIG. 10.

FIG. 7 is a flowchart illustrating an example of an operation of the control apparatus 1-2 according to the present embodiment. Processing in steps S202 to S206 illustrated in FIG. 7 is similar to the processing in steps S102 to S106 described with reference to FIG. 5, and description is thus omitted.

In step S208, the image processing unit 122 determines whether or not the cutout region is about to fall outside the image pickup range. In a case where the cutout region is about to fall outside the image pickup range (YES in step S208), the arm control unit 124 performs the arm control processing such that the position of the mirror portion is brought to the center position in the picked up image (S210), and the process returns to step S202.

On the other hand, in a case where it is not determined that the cutout region is about to fall outside the image pickup range (NO in step S208), the process proceeds to step S212. Processing in steps S212 to S216 illustrated in FIG. 7 is similar to the processing in steps S108 to S112 described with reference to FIG. 5, and description is thus omitted.

The processing in steps S202 to S216 described above may be repeated as appropriate.

FIG. 8 is a flowchart illustrating another example of an operation of the control apparatus 1-2 according to the present embodiment. In the example illustrated in FIG. 8, the arm control processing may be performed all the time irrespective of whether or not the cutout region is about to fall outside the image pickup range.

Processing in steps S222 to S224 illustrated in FIG. 8 is similar to the processing in steps S102 to S104 described with reference to FIG. 5, and description is thus omitted.

In step S226, the arm control unit 124 performs the arm control processing such that the position of the mirror portion is brought to the center position in the picked up image (S226).

Subsequently, the image processing unit 122 performs processing of cutting out the central portion of the picked up image (S228). Processing in subsequent steps S230 to S232 is similar to the processing in steps S110 to S112 described with reference to FIG. 5, and description is thus omitted.

The processing in steps S222 to S232 described above may be repeated as appropriate.

FIG. 9 is a flowchart illustrating another example of an operation of the control apparatus 1-2 according to the present embodiment. In the example illustrated in FIG. 9, the arm control processing is performed such that the image pickup range of the microscope unit 5303 tracks the mirror portion. Further, in the example illustrated in FIG. 9, only the operation related to the arm control processing is illustrated, and image output to the display apparatus 5319 may be performed independently of the present operation. For example, the picked up image of the microscope unit 5303 may be output as it is to the display apparatus 5319 as an output image.

As illustrated in FIG. 9, first, a picked up image is input via the interface unit 30 (S242). Subsequently, it is determined whether or not a mode in which the image pickup range tracks the mirror portion has been brought about (S244). In a case where the mode in which the image pickup range tracks the mirror portion has not been brought about (NO in S244), the process ends.

On the other hand, in a case where the mode in which the image pickup range tracks the mirror portion has been brought about (YES in S244), the mirror portion at the leading end of the dental mirror is detected by the detection unit 20 (S246). Subsequently, the arm control unit 124 performs the arm control processing such that the region of the detected mirror portion is included in the picked up image, and the proportion that the region of the mirror portion occupies in the picked up image becomes larger (S248).

The processing in steps S242 to S248 described above may be repeated as appropriate.

FIG. 10 is a flowchart illustrating another example of an operation of the control apparatus 1-2 according to the present embodiment. In the example illustrated in FIG. 10, the arm control processing is performed such that the image pickup range of the microscope unit 5303 tracks the mirror portion only at a timing instructed by a user, for example.

Further, in the example illustrated in FIG. 10, only the operation related to the arm control processing is illustrated, and image output to the display apparatus 5319 may be performed independently of the present operation. For example, the picked up image of the microscope unit 5303 may be output as it is to the display apparatus 5319 as an output image.

As illustrated in FIG. 10, first, a picked up image is input via the interface unit 30 (S262). Subsequently, it is determined whether or not an instruction that the image pickup range tracks the mirror portion has been made by a user input, for example (S264). In a case where the instruction that the image pickup range tracks the mirror portion has not been made (NO in S264), the process ends.

On the other hand, in a case where the instruction that the image pickup range tracks the mirror portion has been made (YES in S264), processing of detecting the mirror portion at the leading end of the dental mirror is performed by the detection unit 20 (S266).

In step S266, in a case where detection of the mirror portion has succeeded (YES in S268), the arm control unit 124 performs the arm control processing such that the region of the detected mirror portion is included in the picked up image, and the proportion that the region of the mirror portion occupies in the picked up image becomes larger (S270).

In step S266, in a case where detection of the mirror portion has failed (NO in S268), a state such as a distance (for example, a focusing distance) from the observation target is stored in the storage unit 40 (S278). Subsequently, the arm control unit 124 performs the arm control processing such that the image pickup range of the microscope unit 5303 is widened (S280).

Subsequently, the detection unit 20 performs processing of detecting the mirror portion again (S282). In step S282, in a case where detection of the mirror portion has succeeded (YES in S284), the arm control unit 124 performs the arm control processing such that the region of the detected mirror portion is included in the picked up image, and the proportion that the region of the mirror portion occupies in the picked up image becomes larger (S286). Further, the arm control unit 124 performs the arm control processing so as to return to the state stored in step S278 (S292). On the other hand, in a case where detection of the mirror portion has failed in step S282 (NO in S284), the control unit 12 causes an error screen to be output to the display apparatus 5319 (S294), and the process ends.

The processing in steps S262 to S294 described above may be repeated as appropriate.

### (Effects)

The second embodiment of the present disclosure has been described above. In accordance with the second embodiment of the present disclosure, even in a case where the surgical field is wide or a patient has moved, for example, it is possible to perform the arm control processing such that the mirror portion is included in the image pickup range to save the user the effort of setting the image pickup range again manually.

### 2-3. Third Embodiment

Subsequently, as a third embodiment of the present disclosure, an example of using the mirror portion of the dental mirror as a user interface (UI) for the arm control processing will be described.

As described above, the both hands of a user during a dental treatment are occupied by the dental mirror and the surgical instrument, so that the user himself/herself cannot move the arm unit 5309 directly. Therefore, in the present embodiment, the arm control processing is performed such that, when the user moves the position and the angle of the mirror portion of the dental mirror, the microscope unit 5303 performs a pivot movement described with reference to FIG. 1.

### (Exemplary Configuration)

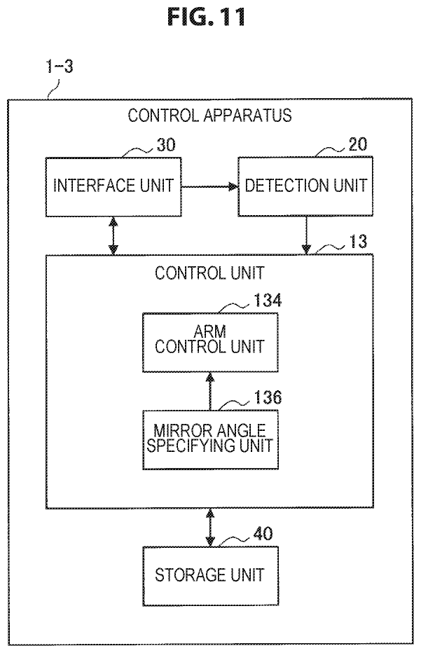

First, an exemplary configuration of the third embodiment of the present disclosure will be described with reference to FIG. 11. FIG. 11 is a block diagram illustrating an example of a functional configuration of a control apparatus 1-3 according to the third embodiment of the present disclosure. The control apparatus 1-3 illustrated in FIG. 11 is equivalent to the control apparatus 5317 described with reference to FIG. 1. As illustrated in FIG. 11, the control apparatus 1-3 includes a control unit 13, the detection unit 20, the interface unit 30, and the storage unit 40. In the components illustrated in FIG. 11, the components of the detection unit 20, the interface unit 30, and the storage unit 40 are substantially identical to the components of the detection unit 20, the interface unit 30, and the storage unit 40 described with reference to FIG. 3, respectively, and description here is thus omitted.

The control unit 13 controls each component of the control apparatus 1-3. Further, the control unit 13 according to the present embodiment also functions as an arm control unit 134 and a mirror angle specifying unit 136 as illustrated in FIG. 11.

The arm control unit 134 performs the arm control processing of controlling the arm unit 5309 described with reference to FIG. 1 and FIG. 2. For example, the arm control unit 134 may perform the arm control processing on the basis of the position of the detected mirror portion. Further, the arm control unit 134 performs the arm control processing on the basis of the angle of the mirror portion specified by the mirror angle specifying unit 136 which will be described later.

For example, the arm control unit 134 may perform the arm control processing such that the microscope unit 5303 translates in accordance with a difference between the position of the mirror portion at a first time (in the past) and the position of the mirror portion at a second time (at present).

Further, the arm control unit 134 may perform the arm control processing such that the microscope unit 5303 performs a pivot movement using the center position of the mirror portion as a pivot axis in accordance with a difference between the angle of the mirror portion at the first time (in the past) and the angle of the mirror portion at the second time (at present).

The above-described translation and pivot movement may be performed at the same time. That is, the microscope unit 5303 may be caused to translate in accordance with the position of the mirror portion detected from the picked up image to capture the mirror portion at the center of the picked up image, and may be caused to perform a pivot movement using the center position of the mirror portion as a pivot axis in accordance with the angle of the mirror portion at that point of time.

With such a configuration, it is possible for the user to observe an affected area reflected on the mirror portion at the first time from another angle by changing the position and the angle of the mirror portion.

The mirror angle specifying unit 136 specifies the angle of the mirror portion detected by the detection unit 20. For example, the mirror angle specifying unit 136 may perform elliptical shape detection by Hough transform processing for the region of the mirror portion detected by the detection unit 20 to specify the angle of the mirror portion on the basis of a curvature (distortion) of a detected elliptical shape.

### (Exemplary Operation)

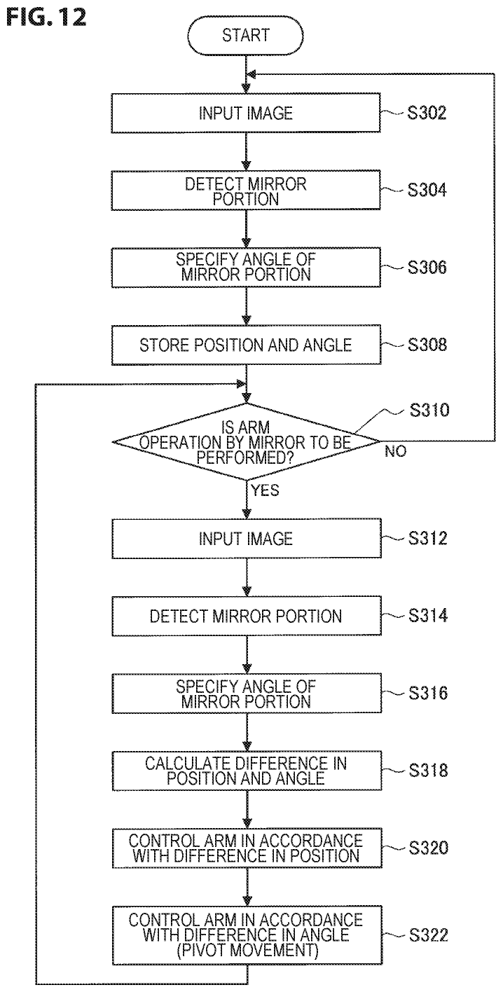

An exemplary configuration of the control apparatus 1-3 according to the present embodiment has been described above. Subsequently, an exemplary operation according to the present embodiment will be described with reference to FIG. 12.

FIG. 12 is a flowchart illustrating an exemplary operation of the control apparatus 1-3 according to the present embodiment. Processing in steps S302 to S304 illustrated in FIG. 12 is similar to the processing in steps S102 to S104 described with reference to FIG. 5, and description is thus omitted.

Subsequently, the mirror angle specifying unit 136 specifies the angle of the mirror portion detected in step S304 (S306). Subsequently, in step S308, the position and the angle of the mirror portion at the present time (the first time) are stored in the storage unit 40 (S308). It is to be noted that, in step S308, the affected area (observation target) is included in the mirror portion in the picked up image, and a trigger for storing the position and the angle of the mirror portion may be a user input, for example.

Subsequently, it is determined whether or not to perform an arm operation by the dental mirror (S310). The determination in step S310 may be performed on the basis of a user input or previously-set mode setting of the medical observation system 5300, for example. In a case where an arm operation by the dental mirror is not to be performed (NO in S310), the process returns to step S302.

Subsequently, in a case where an arm operation by the dental mirror is to be performed (YES in S310), the process proceeds to step S312. Processing in steps S312 to S316 is similar to the processing in steps S302 to S306, and description is thus omitted.

Subsequently, a difference between the position and the angle of the mirror portion at the first time stored in step S308 and the position and the angle of the mirror portion at present (the second time) is calculated (S318).

Subsequently, the arm control unit 134 performs the arm control processing such that the microscope unit 5303 translates in accordance with the difference in position of the mirror portion calculated in step S318 (S320). Furthermore, the arm control unit 134 performs the arm control processing such that the microscope unit 5303 performs a pivot movement in accordance with the difference in angle of the mirror portion calculated in step S318 (S322). Subsequently, the process returns to step S310.

### (Effects)

The third embodiment of the present disclosure has been described above. In accordance with the third embodiment of the present disclosure, it is possible for the user to observe an affected area from another angle by changing the position and the angle of the mirror portion. Such observation is equivalent to, in observation with the naked eyes, for example, observation by the user while moving the head to change the angle, and more intuitive observation for the user may be achieved.

In accordance with the present embodiment, in a case where, when the user changes the position of a surgical instrument such as a drill, for example, the surgical instrument interferes with the line of sight of the microscope unit 5303 so that a desired field of view cannot be obtained, it is possible to move the microscope unit 5303 to obtain the desired field of view by adjusting the position and the angle of the mirror portion.

### 2-4. Fourth Embodiment

Subsequently, as a fourth embodiment, an example where navigation information that specifies such a suitable angle of the mirror portion that an observation target is included in the mirror portion, and guides a user to achieve the suitable angle is displayed will be described. In accordance with the present embodiment, in a case where the position and the attitude of the microscope unit 5303 have been changed for some reason, and an affected area can no longer be observed, navigation information for observing the affected area without further changing the position and the attitude of the microscope unit 5303 at present may be provided for the user. For example, it is possible for the user to observe the affected area by changing the angle of the mirror portion in accordance with the navigation information.

### (Exemplary Configuration)

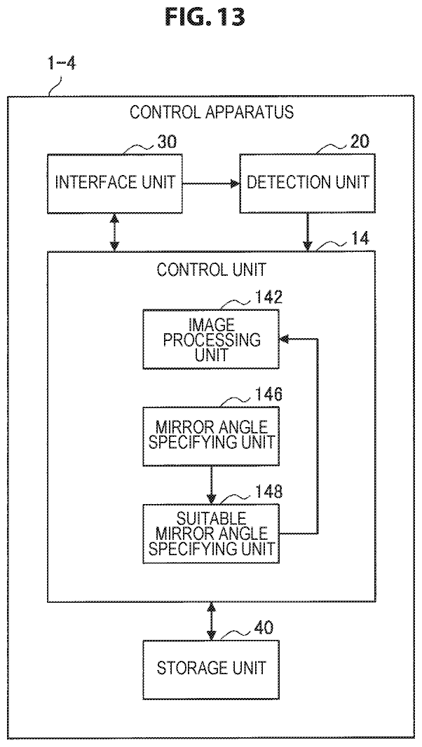

First, an exemplary configuration of the fourth embodiment of the present disclosure will be described with reference to FIG. 13 to FIG. 17. FIG. 13 is a block diagram illustrating an example of a functional configuration of a control apparatus 1-4 according to the fourth embodiment of the present disclosure. The control apparatus 1-4 illustrated in FIG. 13 is equivalent to the control apparatus 5317 described with reference to FIG. 1. As illustrated in FIG. 13, the control apparatus 1-4 includes a control unit 14, the detection unit 20, the interface unit 30, and the storage unit 40. In the components illustrated in FIG. 13, the components of the detection unit 20, the interface unit 30, and the storage unit 40 are substantially identical to the components of the detection unit 20, the interface unit 30, and the storage unit 40 described with reference to FIG. 3, respectively, and description here is thus omitted.

The control unit 14 controls each component of the control apparatus 1-4. Further, the control unit 14 according to the present embodiment also functions as an image processing unit 142, a mirror angle specifying unit 146, and a suitable mirror angle specifying unit 148 as illustrated in FIG. 13.

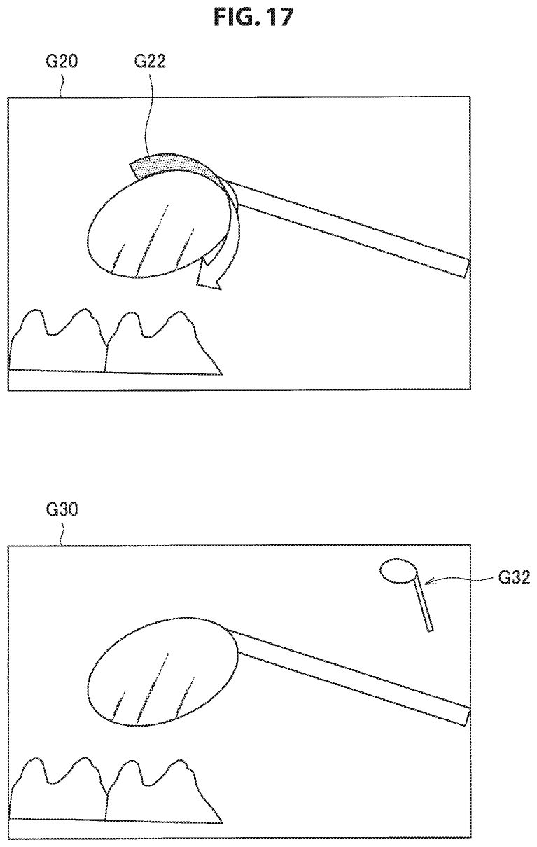

The image processing unit 142 performs image processing on a picked up image on the basis of the dental mirror detection information provided from the detection unit 20. The image processing unit 142 according to the present embodiment performs image processing of combining navigation information for guidance to a suitable angle of the mirror portion specified by the suitable mirror angle specifying unit 148 which will be described later into a picked up image. For example, the image processing unit 142 may superimpose the navigation information over the picked up image to generate an output image. It is to be noted that an example of navigation information will be described later with reference to FIG. 17.

The mirror angle specifying unit 146 specifies the angle of the mirror portion detected by the detection unit 20, similarly to the mirror angle specifying unit 136 described with reference to FIG. 11. Further, the suitable mirror angle specifying unit 148 specifies a suitable angle of the mirror portion such that an observation target (for example, affected area) is included in the mirror portion in the picked up image on the basis of the angle of the mirror portion specified by the mirror angle specifying unit 146. The suitable mirror angle specifying unit 148 specifies a suitable angle of the mirror portion further on the basis of camera parameters (position and attitude) related to the microscope unit 5303. It is to be noted that camera parameters related to the microscope unit 5303 may be obtained on the basis of information about each joint angle and length of the arm unit 5309, for example, or may be sensed and obtained by an optical sensor or a magnetic sensor with an optical or magnetic marker attached to the microscope unit 5303.

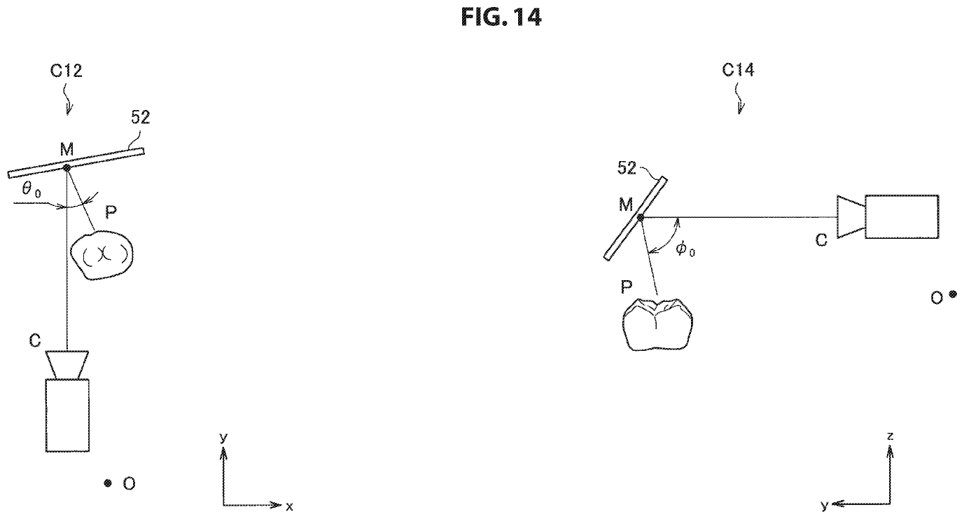

Hereinafter, specification of a suitable angle performed by the suitable mirror angle specifying unit 148 will be described with reference to FIG. 14 to FIG. 16.

FIG. 14 illustrates an initial state where an affected area (observation target) existing at a position P can be observed. Further, in a plan view C12 and a side view C14 illustrated in FIG. 14, relative coordinates at a position C and the attitude of a camera (the microscope unit 5303) with respect to the origin O have been obtained. Furthermore, a distance CM from the camera to the mirror portion 52, a focusing distance CM+MP from the camera to the affected area, angles θ₀ and φ₀ of the mirror portion can be observed. It is possible to obtain relative coordinates of a position P of the affected area with respect to the origin O from the above-described observable information.

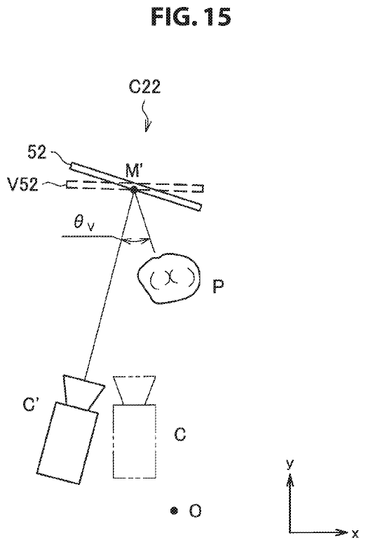

FIG. 15 is a plan view C22 in a state where the position and the attitude of the camera have been changed from the state illustrated in FIG. 14. Here, in a case where the relative relationship between the origin O and the position P of the affected area is invariable, the suitable mirror angle specifying unit 148 can specify a suitable angle θᵥ of the mirror portion from a camera position C′ after the movement, a distance C′M to the mirror portion 52, and the position P of the affected area. It is to be noted that the attitude of the mirror portion at the suitable angle is illustrated in FIG. 15 as a reference character V52.

Further, since the present angle of the mirror portion 52 is specified by the mirror angle specifying unit 146, it is also possible to calculate an angular difference between the mirror portion 52 and the mirror portion V52 at the suitable angle. It is to be noted that an example of calculating an angular difference around the Z-axis in the plan view has been described above, whilst it is also possible to similarly calculate an angular difference around the X-axis in the side view.

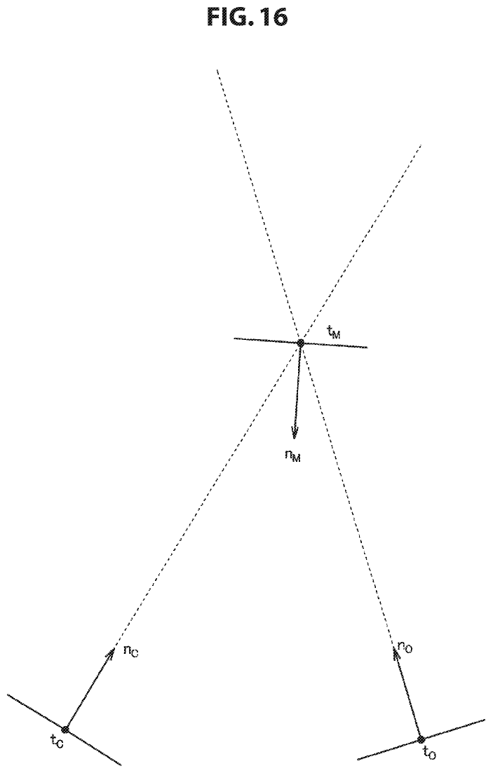

FIG. 16 is an explanatory diagram for describing a procedure for calculating the suitable angle in more detail. In FIG. 16, the position and the orientation of each of the camera, the mirror portion at the suitable angle, and the observation target are expressed by (t,n)t,n∈R³. Further, n_{C} indicates a camera optical axis, and n_{M}, n_{O} indicate normal directions of the mirror portion and the observation target at the suitable angle, respectively, and assume the following holds.

[Math. 1]

|n_{C}|=|n_{M}|=|n_{O}|=1

Further, assuming that an optical path length from the camera to the observation target via the mirror portion is equal to a focusing distance l of the camera, the following relationship is obtained.

[Math. 2]

|\vec{t_{C}t_{M}}|+|\vec{t_{M}t_{O}}|=l

Here, assuming that the following hold,

[Math. 3]

\vec{t_{C}t_{M}}=t_{C}+an_{C},|\vec{t_{C}t_{M}}|=a

\vec{t_{O}t_{M}}=t_{O}+bn_{O},|\vec{t_{O}t_{M}}|=b

a relationship of Formula (1) below is obtained.

[Math. 4]

a+b=l (1)

Further, the position t_{M} of the mirror portion is an intersection of the following two vectors.

[Math. 5]

\vec{t_{C}t_{M}}

\vec{t_{O}t_{M}}

Consequently, a relationship of Formula (2) below is obtained.

[Math. 6]

t_{M}=t_{C}+an_{C}=t_{O}+bn_{O} (2)

From Formula (1) and Formula (2) above, a and b are obtained in the following manner, respectively.

$$\begin{matrix}[ Math . 7 ] & \\ {t}_{C} + {an}_{C} = {t}_{O} + ( l - a ) {n}_{O} \\ a = \frac{{t}_{O} - {t}_{C} + {ln}_{O}}{{n}_{C} + {n}_{O}} , b = \frac{{t}_{C} - {t}_{o} + l {n}_{C}}{{n}_{C} + {n}_{O}} & \end{matrix}$$

Further, the position t_{M} of the mirror portion is obtained by Formula (3) below.

$$\begin{matrix}[ Math . 8 ] & \\ {t}_{M} = \frac{{t}_{O} {n}_{C} + {t}_{C} {n}_{o} + l {n}_{C} {n}_{O}}{{n}_{C} + {n}_{O}} & ( 3 )\end{matrix}$$

Further, since the angle of incidence and the angle of reflection with respect to the mirror portion are equal, an orientation (suitable angle) n_{M} of the mirror portion in which the observation target can be observed is expressed by Formula (4) below.

$$\begin{matrix}[ Math . 9 ] & \\ {n}_{M} = - \frac{{n}_{C} + {n}_{O}}{2} & ( 4 )\end{matrix}$$

As described above, the suitable mirror angle specifying unit 148 may specify a suitable angle of the mirror portion.

FIG. 17 includes explanatory diagrams illustrating an example of navigation information for guidance to a suitable angle described above. For example, the image processing unit 142 may combine navigation information G22 which is an arrow indicating a rotation direction of guiding the present mirror portion to a suitable angle into the picked up image as in an output image G20 illustrated in FIG. 17. Further, the image processing unit 142 may combine navigation information G32 indicating an image of the dental mirror at the suitable angle into the picked up image as in an output image G30 illustrated in FIG. 17.

When an output image as illustrated in FIG. 17 in which navigation information for guidance to a suitable angle has been combined into a picked up image is displayed, it is possible for the user to adjust the mirror portion to the suitable angle, and to observe the affected area (observation target). It is to be noted that the example illustrated in FIG. 17 is a mere example, and navigation information is not limited to the example of FIG. 17. For example, the navigation information may include a suitable angle, numerical information indicating a difference between a suitable angle and the present angle of the mirror portion, and the like. Further, the navigation information may include animation information of an arrow indicating a rotation direction and animation information in which the angle of an image of the dental mirror changes.

### (Exemplary Operation)

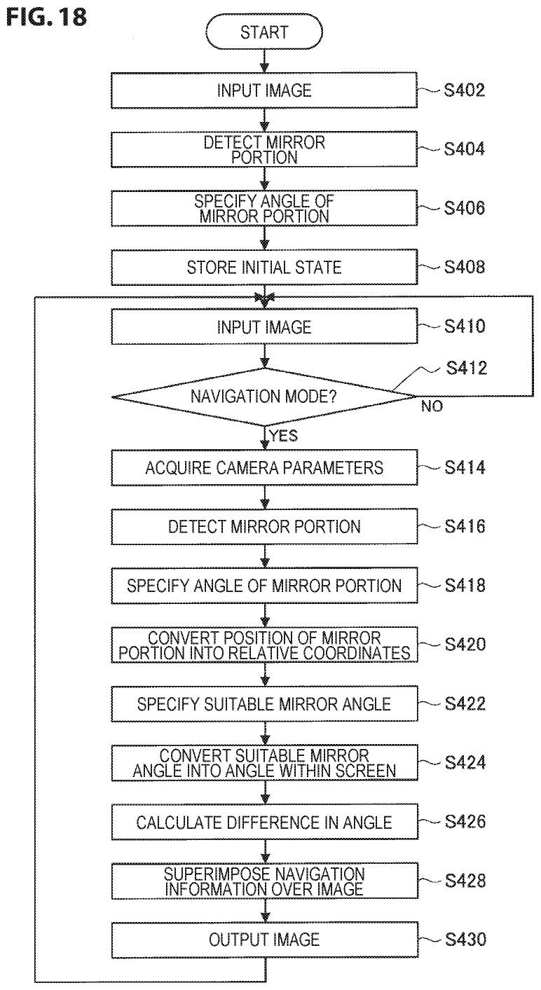

An exemplary configuration of the control apparatus 1-4 according to the present embodiment has been described above. Subsequently, an exemplary operation according to the present embodiment will be described with reference to FIG. 18.

FIG. 18 is a flowchart illustrating an exemplary operation of the control apparatus 1-4 according to the present embodiment. Processing in steps S402 to S406 illustrated in FIG. 18 is similar to the processing in steps S302 to S306 described with reference to FIG. 12, and description is thus omitted.

Subsequently, in step S408, information about an initial state described with reference to FIG. 14 is stored in the storage unit 40. It is to be noted that, in step S408, the affected area (observation target) is included in the mirror portion in the picked up image, and a trigger for storing the initial state may be a user input, for example.

Subsequently, a picked up image is input via the interface unit 30 (S410). Subsequently, it is determined whether or not a mode of causing the navigation information to be output has been brought about (S412). The determination in step S412 may be performed on the basis of previously-set mode setting of the medical observation system 5300, or may be performed on the basis of a user input. In a case where the mode of causing the navigation information to be output has not been brought about (NO in S412), the process returns to step S410.

In a case where it is determined that the mode of causing the navigation information to be output has been brought about (YES in S412), the camera parameters (position and attitude) related to the microscope unit 5303 at the present time are acquired (S414). It is to be noted that the camera parameters related to the microscope unit 5303 in step S414 may be different from the camera parameters related to the microscope unit 5303 stored in step S408 as an initial state.

Since processing in subsequent steps S416 to S418 is similar to the processing in steps S404 to S406, description is omitted. Subsequently, the position of the mirror portion detected in step S416 is converted into relative coordinates with respect to the origin O described with reference to FIG. 14 and FIG. 15 (S420).

Subsequently, as described with reference to FIG. 14 to FIG. 16, for example, the suitable mirror angle specifying unit 148 specifies a suitable angle (suitable mirror angle) of the mirror portion at the relative coordinates with respect to the origin O (S422). Furthermore, the suitable mirror angle specifying unit 148 converts the suitable angle obtained in step S422 into an angle (an angle in the picked up image) within the screen (S424). Subsequently, a difference between the suitable angle of the mirror portion obtained in step S424 and the angle of the mirror portion specified in step S418 is calculated (S426).

Subsequently, the image processing unit 142 performs image processing of superimposing the navigation information for guidance to a suitable angle over the picked up image on the basis of the angular difference obtained in step S426 (S428). An output image obtained by superposition in step S428 is output to the display apparatus 5319 (S430). Subsequently, the process returns to step S410.

### (Effects)

The fourth embodiment of the present disclosure has been described above. In accordance with the fourth embodiment of the present disclosure, in a case where the position and the attitude of the microscope unit 5303 are changed for some reason, and the affected area can no longer be observed, navigation information for observing the affected area without further changing the position and the attitude of the microscope unit 5303 at present can be displayed. Further, it is possible for the user to observe the affected area by changing the angle of the mirror portion in accordance with the navigation information.

It is to be noted that, since the control apparatus 1-4 according to the present embodiment may not perform the arm control processing, the present embodiment can be applied even in a case where the microscope apparatus 5301 illustrated in FIG. 1 does not include the driving mechanism of the arm unit 5309.

### 2-5. Fifth Embodiment

In the fourth embodiment described above, an example of providing the user with navigation information for guiding the user such that the affected area (observation target) can be observed without changing the position and the attitude of the camera (the microscope unit 5303) at present has been described.

Subsequently, as a fifth embodiment, an example of performing the arm control processing so as to obtain the position and the attitude of the camera at which an affected area can be observed in accordance with the position and the attitude of the mirror portion at present using both the affected area and the mirror portion as references will be described.

### (Exemplary Configuration)

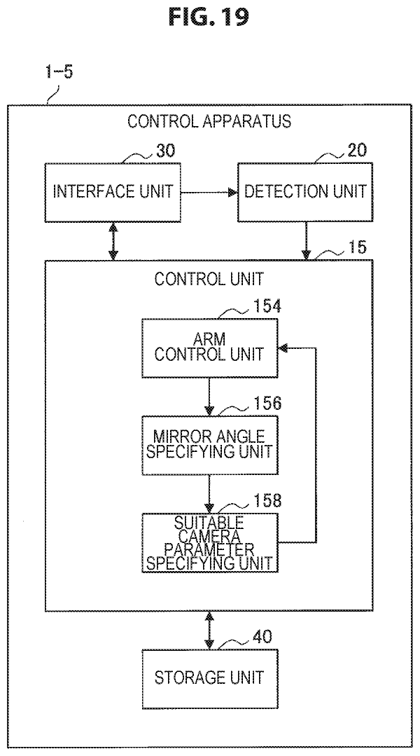

First, an exemplary configuration of the fifth embodiment of the present disclosure will be described with reference to FIG. 19. FIG. 19 is a block diagram illustrating an example of a functional configuration of a control apparatus 1-5 according to the fifth embodiment of the present disclosure. The control apparatus 1-5 illustrated in FIG. 19 is equivalent to the control apparatus 5317 described with reference to FIG. 1. As illustrated in FIG. 19, the control apparatus 1-5 includes a control unit 15, the detection unit 20, the interface unit 30, and the storage unit 40. In the components illustrated in FIG. 19, the components of the detection unit 20, the interface unit 30, and the storage unit 40 are substantially identical to the components of the detection unit 20, the interface unit 30, and the storage unit 40 described with reference to FIG. 3, respectively, and description here is thus omitted.

The control unit 15 controls each component of the control apparatus 1-5. Further, the control unit 15 according to the present embodiment also functions as an arm control unit 154, a mirror angle specifying unit 156, and a suitable camera parameter specifying unit 158 as illustrated in FIG. 19.

The arm control unit 154 performs the arm control processing of controlling the arm unit 5309 described with reference to FIG. 1 and FIG. 2. The arm control unit 154 according to the present embodiment performs the arm control processing such that, on the basis of the camera parameters (position and attitude) related to the microscope unit 5303 specified by the suitable camera parameter specifying unit 158 which will be described later, the camera parameters are achieved, for example.

The mirror angle specifying unit 156 specifies the angle of the mirror portion detected by the detection unit 20 similarly to the mirror angle specifying unit 136 described with reference to FIG. 11.

The suitable camera parameter specifying unit 158 specifies such camera parameters (hereinafter called suitable camera parameters) related to the microscope unit 5303 that an observation target is included in the mirror portion in the picked up image on the basis of the angle of the mirror portion specified by the mirror angle specifying unit 156.

Here, specification of the suitable camera parameters performed by the suitable camera parameter specifying unit 158 will be described with reference to FIG. 16.

From Formula (4) derived with reference to FIG. 16, an orientation (attitude) n_{C} of the camera included in suitable camera parameters is calculated as in Formula (5) below.

[Math. 10]

n_{C}=−(n_{O}+2n_{M}) (5)

From Formula (3) derived with reference to FIG. 16 and Formula (5) above, a camera position t_{C} included in the suitable camera parameters is calculated as in the formula below.

$$\begin{matrix}[ Math . 11 ] & \\ {t}_{C} = \frac{{n}_{C} ( {t}_{M} - {t}_{O} - l {n}_{O} )}{{n}_{O}} + {t}_{M} & \end{matrix}$$

As described above, the suitable camera parameter specifying unit 158 may specify suitable camera parameters (position and attitude) related to the microscope unit 5303.

### (Exemplary Operation)

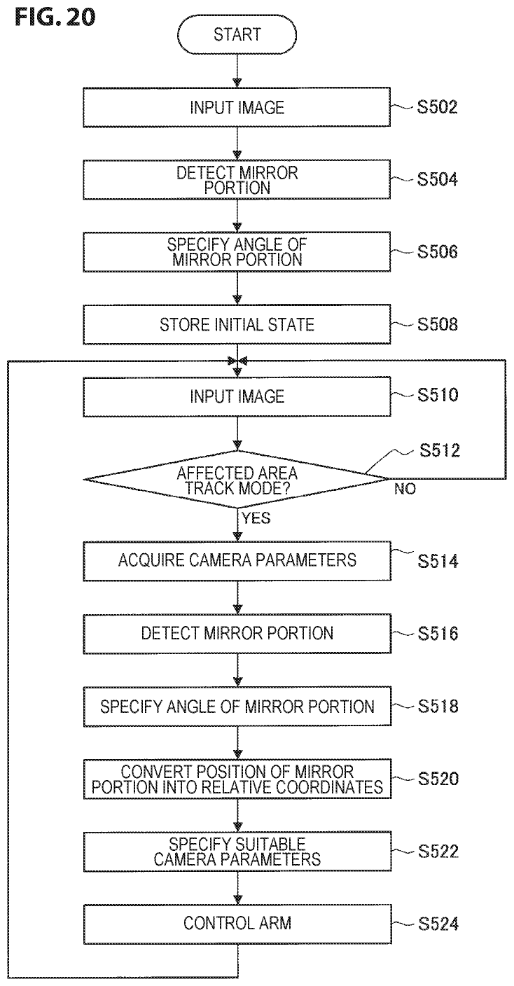

An exemplary configuration of the control apparatus 1-5 according to the present embodiment has been described above. Subsequently, an exemplary operation according to the present embodiment will be described with reference to FIG. 20. FIG. 20 is a flowchart illustrating an exemplary operation of the control apparatus 1-5 according to the present embodiment. It is to be noted that, in the example illustrated in FIG. 20, only the operation related to the arm control processing is illustrated, and image output to the display apparatus 5319 may be performed independently of the present operation. For example, the picked up image of the microscope unit 5303 may be output as it is to the display apparatus 5319 as an output image.

Processing in steps S502 to S510 illustrated in FIG. 20 is similar to the processing in steps S402 to S410 described with reference to FIG. 18, and description is thus omitted.

Subsequently, it is determined whether or not a mode in which the camera tracks an affected area (observation target) has been brought about (S512). The determination in step S512 may be performed on the basis of previously-set mode setting of the medical observation system 5300, or may be performed on the basis of a user input. In a case where the mode in which the camera tracks an affected area has not been brought about (NO in S512), the process returns to step S510.

In a case where it is determined that the mode in which the camera tracks an affected area has been brought about (YES in S512), the process proceeds to step S514. Since processing in subsequent steps S514 to S520 is similar to the processing in steps S414 to S420 described with reference to FIG. 18, description is omitted.

The suitable camera parameter specifying unit 158 specifies suitable camera parameters (S522). Furthermore, the arm control unit 154 performs the arm control processing on the basis of the suitable camera parameters (S524). Subsequently, the process returns to step S510.

### (Effects)

The fifth embodiment of the present disclosure has been described above. In accordance with the fifth embodiment of the present disclosure, the position and the attitude of the microscope unit 5303 are automatically controlled such that, even in a case where the user has changed the position and the angle of the mirror portion, an affected area can be observed. Consequently, it is possible for the user to observe the affected area while changing the position and the angle of the mirror portion more intuitively, without being aware of the position and the attitude of the microscope unit 5303.

### 3. HARDWARE CONFIGURATION EXAMPLE

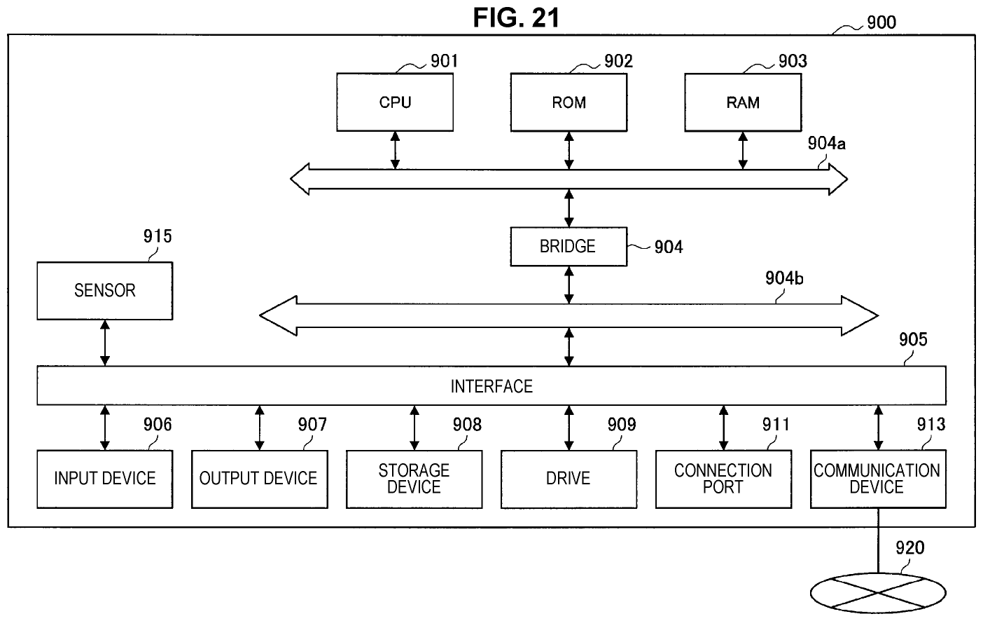

The embodiment of the present disclosure has been described hitherto. Finally, a hardware configuration of an information processing apparatus according to the present embodiment of the present disclosure will be described with reference to FIG. 21. FIG. 21 is a block diagram illustrating an example of the hardware configuration of the information processing apparatus according to the present embodiment of the present disclosure. Meanwhile, an information processing apparatus 900 illustrated in FIG. 21 may realize the control apparatus 1-1, the control apparatus 1-2, the control apparatus 1-3, the control apparatus 1-4, and the control apparatus 1-5, for example. Information processing by the control apparatus 1-1, the control apparatus 1-2, the control apparatus 1-3, the control apparatus 1-4, and the control apparatus 1-5 according to the present embodiment is realized according to cooperation between software and hardware described below.

As illustrated in FIG. 21, the information processing apparatus 900 includes a central processing unit (CPU) 901, a read only memory (ROM) 902, a random access memory (RAM) 903, and a host bus 904a. In addition, the information processing apparatus 900 includes a bridge 904, an external bus 904b, an interface 905, an input device 906, an output device 907, a storage device 908, a drive 909, a connection port 911, a communication device 913, and a sensor 915. The information processing apparatus 900 may include a processing circuit such as a DSP or an ASIC instead of the CPU 901 or along therewith.

The CPU 901 functions as an arithmetic processing device and a control device and controls the overall operation in the information processing apparatus 900 according to various programs. Further, the CPU 901 may be a microprocessor. The ROM 902 stores programs, operation parameters, and the like used by the CPU 901. The RAM 903 temporarily stores programs used in execution of the CPU 901, parameters appropriately changed in the execution, and the like. The CPU 901 may form the control unit 11, the control unit 12, the control unit 13, the control unit 14, and the control unit 15, for example.

The CPU 901, the ROM 902, and the RAM 903 are connected by the host bus 904a including a CPU bus and the like. The host bus 904a is connected with the external bus 904b such as a peripheral component interconnect/interface (PCI) bus via the bridge 904. Further, the host bus 904a, the bridge 904, and the external bus 904b are not necessarily separately configured and such functions may be mounted in a single bus.

The input device 906 is realized by a device through which a user inputs information, such as a mouse, a keyboard, a touch panel, a button, a microphone, a switch, and a lever, for example. In addition, the input device 906 may be a remote control device using infrared ray or other electric waves, or external connection equipment such as a cellular phone or a PDA corresponding to an operation of the information processing apparatus 900, for example. Furthermore, the input device 906 may include an input control circuit or the like which generates an input signal on the basis of information input by the user using the aforementioned input means and outputs the input signal to the CPU 901, for example. The user of the information processing apparatus 900) may input various types of data or order a processing operation for the information processing apparatus 900 by operating the input device 906.

The output device 907 is formed by a device that may visually or aurally notify the user of acquired information. As such devices, there are a display device such as a CRT display device, a liquid crystal display device, a plasma display device, an EL display device, or a lamp, a sound output device such as a speaker and a headphone, a printer device, and the like. The output device 907 outputs results acquired through various processes performed by the information processing apparatus 900, for example. Specifically, the display device visually displays results acquired through various processes performed by the information processing apparatus 900 in various forms such as text, images, tables, and graphs. On the other hand, the sound output device converts audio signals including reproduced sound data, audio data, and the like into analog signals and aurally outputs the analog signals.

The storage device 908 is a device for data storage, formed as an example of a storage unit of the information processing apparatus 900. For example, the storage device 908 is realized by a magnetic storage device such as an HDD, a semiconductor storage device, an optical storage device, a magneto-optical storage device, or the like. The storage device 908 may include a storage medium, a recording device for recording data on the storage medium, a reading device for reading data from the storage medium, a deletion device for deleting data recorded on the storage medium, and the like. The storage device 908 stores programs and various types of data executed by the CPU 901, various types of data acquired from the outside, and the like. The storage device 908 may form the storage unit 40, for example.

The drive 909 is a reader/writer for storage media and is included in or externally attached to the information processing apparatus 900. The drive 909 reads information recorded on a removable storage medium such as a magnetic disc, an optical disc, a magneto-optical disc, or a semiconductor memory mounted thereon, and outputs the information to the RAM 903. In addition, the drive 909 may write information regarding the removable storage medium.

The connection port 911 is an interface connected with external equipment and is a connector to the external equipment through which data may be transmitted through a universal serial bus (USB) and the like, for example.

The communication device 913 is a communication interface formed by a communication device for connection to a network 920 or the like, for example. The communication device 913 is a communication card or the like for a wired or wireless local area network (LAN), long term evolution (LTE). Bluetooth (registered trademark), or wireless USB (WUSB), for example. In addition, the communication device 913 may be a router for optical communication, a router for asymmetric digital subscriber line (ADSL), various communication modems, or the like. For example, the communication device 913 may transmit/receive signals and the like to/from the Internet and other communication apparatuses according to a predetermined protocol such as, for example, TCP/IP.

The sensor 915 corresponds to various types of sensors such as an acceleration sensor, a gyro sensor, a geomagnetic sensor, a light sensor, a sound sensor, a distance measuring sensor, and a force sensor, for example. The sensor 915 acquires information regarding a state of the information processing apparatus 900 itself, such as an attitude and a movement speed of the information processing apparatus 900, and information regarding a surrounding environment of the information processing apparatus 900, such as brightness and noise of the periphery of the information processing apparatus 900. In addition, the sensor 915 may include a GPS sensor that receives a GPS signal, and measures latitude, longitude, and altitude of the device.

Further, the network 920 is a wired or wireless transmission path of information transmitted from devices connected to the network 920. For example, the network 920 may include a public circuit network such as the Internet, a telephone circuit network, or a satellite communication network, various local area networks (LANs) including Ethernet (registered trademark), a wide area network (WAN), and the like. In addition, the network 920 may include a dedicated circuit network such as an internet protocol-virtual private network (IP-VPN).

Hereinbefore, an example of a hardware configuration capable of realizing the functions of the information processing apparatus 900 according to this embodiment is shown. The respective components may be implemented using universal members, or may be implemented by hardware specific to the functions of the respective components. Accordingly, according to a technical level at the time when the embodiments are executed, it is possible to appropriately change hardware configurations to be used.

In addition, a computer program for realizing each of the functions of the information processing apparatus 900 according to the present embodiment as described above may be created, and may be mounted in a PC or the like. Furthermore, a computer-readable recording medium on which such a computer program is stored may be provided. The recording medium is a magnetic disc, an optical disc, a magneto-optical disc, a flash memory, or the like, for example. Further, the computer program may be delivered through a network, for example, without using the recording medium. In addition, the above-described computer program may be distributed through, for example, a network without using a recording medium.

### 4. CONCLUSION

In accordance with the embodiments of the present disclosure as described above, it is possible to observe an affected area (observation target) more freely.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

For example, in the above-described embodiments, an example where the detection unit included in the control apparatus detects the mirror portion of the dental mirror has been described, whilst the present technology is not limited to such an example, but detection of the mirror portion may be performed in an apparatus other than the control apparatus. Further, in such a case, the interface unit, for example, may function as an acquisition unit to receive and acquire dental mirror detection information from another apparatus.

Further, the control unit included in the control apparatus may further control the optical magnification (focusing distance) of the microscope unit 5303. For example, in the second embodiment described above, an example of performing the arm control processing such that the image pickup range of the microscope unit 5303 is widened has been described, whilst the control unit may widen the image pickup range by controlling the above-described optical magnification in addition to or instead of the arm control processing.

Further, the control unit included in the control apparatus may perform image processing of superimposing alarm information that notifies a user of an alarm. For example, in the second embodiment described above, in a case where it is determined that a cutout region or the mirror portion is about to fall outside the image pickup range, alarm information that notifies an alarm indicating that the cutout region or the mirror portion is about to fall outside the image pickup range may be superimposed over the picked up image. With such a configuration, it is possible for the user to adjust the position and the angle of the mirror portion such that the cutout region or the mirror portion does not fall outside the image pickup range.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1)

A control apparatus including:
an acquisition unit configured to acquire dental mirror detection information from a picked up image acquired by an image pickup apparatus, and
a control unit configured to, on the basis of the dental mirror detection information, perform at least one of image processing on the picked up image or arm control processing that controls an arm unit that supports the image pickup apparatus.
   (2)

The control apparatus according to (1), in which
the control unit at least performs the image processing, and
the image processing includes processing of cutting out a cutout region specified on the basis of the dental mirror detection information.
   (3)

The control apparatus according to (2), in which
the dental mirror detection information includes information indicating a region of a mirror portion of a detected dental mirror, and
the cutout region includes the region of the mirror portion.
   (4)

The control apparatus according to any one of (1) to (3), in which
the dental mirror detection information includes information indicating a position of a mirror portion of a detected dental mirror, and
the control unit performs the arm control processing on the basis of the position of the mirror portion.
   (5)

The control apparatus according to (4), in which
the control unit performs the arm control processing such that the position of the mirror portion is brought to a predetermined position on the picked up image.
   (6)

The control apparatus according to (5), in which
the dental mirror detection information further includes information indicating a region of the mirror portion, and
the control unit performs the arm control processing in a case where at least part of a region of the dental mirror overlaps an outer peripheral region of the picked up image.
   (7)

The control apparatus according to (4), in which
the dental mirror detection information further includes information indicating a region of the mirror portion, and
the control unit performs the arm control processing such that a region of the dental mirror is included and a proportion that the region of the dental mirror occupies in the picked up image becomes larger.
   (8)

The control apparatus according to (7), in which
the dental mirror detection information further includes information indicating whether or not detection of the mirror portion of the dental mirror has succeeded, and
the control unit performs the arm control processing such that, in a case where information indicating that detection of the mirror portion has failed is included in the dental mirror detection information, an image pickup range of the image pickup apparatus is widened.
   (9)

The control apparatus according to (1), in which
the control unit performs the arm control processing on the basis of an angle of a mirror portion of a dental mirror detected from the picked up image.
   (10)

The control apparatus according to (9), in which
the control unit performs the arm control processing in accordance with a difference between the angle of the mirror portion at a first time and the angle of the mirror portion at a second time.
   (11)

The control apparatus according to (9), in which
the control unit specifies such a camera parameter related to the image pickup apparatus that an observation target is included in the mirror portion on the picked up image on the basis of the angle of the mirror portion, and performs the arm control processing on the basis of the camera parameter.
   (12)

The control apparatus according to (1), in which
the control unit specifies such a suitable angle of a mirror portion that an observation target is included in the mirror portion of a dental mirror in the picked up image, and performs the image processing of combining navigation information for guidance to the suitable angle into the picked up image.
   (13)

A control method including:
acquiring dental mirror detection information from a picked up image acquired by an image pickup apparatus; and
on the basis of the dental mirror detection information, performing, with a processor, at least one of image processing on the picked up image or arm control processing that controls an arm unit that supports the image pickup apparatus.
   (14)

A medical observation system including:
an image pickup apparatus configured to acquire a picked up image; and
a control apparatus including
   an acquisition unit configured to acquire dental mirror detection information from the picked up image, and
   a control unit configured to, on the basis of the dental mirror detection information, perform at least one of image processing on the picked up image or arm control processing that controls an arm unit that supports the image pickup apparatus.

### REFERENCE SIGNS LIST

1 control apparatus
5 dental mirror
11, 12, 13, 14, 15 control unit
20 detection unit
30 interface unit
40 storage unit
52 mirror portion
112 image processing unit
124 arm control unit
136 mirror angle specifying unit
148 suitable mirror angle specifying unit
158 suitable camera parameter specifying unit

1. A control apparatus comprising:
an acquisition unit configured to acquire dental mirror detection information from a picked up image acquired by an image pickup apparatus; and
a control unit configured to, on a basis of the dental mirror detection information, perform at least one of image processing on the picked up image or arm control processing that controls an arm unit that supports the image pickup apparatus.

2. The control apparatus according to claim 1, wherein
the control unit at least performs the image processing, and
the image processing includes processing of cutting out a cutout region specified on a basis of the dental mirror detection information.

3. The control apparatus according to claim 2, wherein
the dental mirror detection information includes information indicating a region of a mirror portion of a detected dental mirror, and
the cutout region includes the region of the mirror portion.

4. The control apparatus according to claim 1, wherein
the dental mirror detection information includes information indicating a position of a mirror portion of a detected dental mirror, and
the control unit performs the arm control processing on a basis of the position of the mirror portion.

5. The control apparatus according to claim 4, wherein
the control unit performs the arm control processing such that the position of the mirror portion is brought to a predetermined position on the picked up image.

6. The control apparatus according to claim 5, wherein
the dental mirror detection information further includes information indicating a region of the mirror portion, and
the control unit performs the arm control processing in a case where at least part of a region of the dental mirror overlaps an outer peripheral region of the picked up image.

7. The control apparatus according to claim 4, wherein
the dental mirror detection information further includes information indicating a region of the mirror portion, and
the control unit performs the arm control processing such that a region of the dental mirror is included and a proportion that the region of the dental mirror occupies in the picked up image becomes larger.

8. The control apparatus according to claim 7, wherein
the dental mirror detection information further includes information indicating whether or not detection of the mirror portion of the dental mirror has succeeded, and
the control unit performs the arm control processing such that, in a case where information indicating that detection of the mirror portion has failed is included in the dental mirror detection information, an image pickup range of the image pickup apparatus is widened.

9. The control apparatus according to claim 1, wherein
the control unit performs the arm control processing on a basis of an angle of a mirror portion of a dental mirror detected from the picked up image.

10. The control apparatus according to claim 9, wherein
the control unit performs the arm control processing in accordance with a difference between the angle of the mirror portion at a first time and the angle of the mirror portion at a second time.

11. The control apparatus according to claim 9, wherein
the control unit specifies such a camera parameter related to the image pickup apparatus that an observation target is included in the mirror portion on the picked up image on a basis of the angle of the mirror portion, and performs the arm control processing on a basis of the camera parameter.

12. The control apparatus according to claim 1, wherein
the control unit specifies such a suitable angle of a mirror portion that an observation target is included in the mirror portion of a dental mirror in the picked up image, and performs the image processing of combining navigation information for guidance to the suitable angle into the picked up image.

13. A control method comprising:
acquiring dental mirror detection information from a picked up image acquired by an image pickup apparatus; and
on a basis of the dental mirror detection information, performing, with a processor, at least one of image processing on the picked up image or arm control processing that controls an arm unit that supports the image pickup apparatus.

14. A medical observation system comprising:
an image pickup apparatus configured to acquire a picked up image; and
a control apparatus including
an acquisition unit configured to acquire dental mirror detection information from the picked up image, and
a control unit configured to, on a basis of the dental mirror detection information, perform at least one of image processing on the picked up image or arm control processing that controls an arm unit that supports the image pickup apparatus.
